(19) United States
(12) Patent Application Publication
(10) Pub. No.: US 2013/0029873 A1
(43) Pub. Date: Jan. 31, 2013

(54) METHODS AND COMPOSITIONS FOR DIAGNOSING PULMONARY FIBROSIS SUBTYPES AND ASSESSING THE RISK OF PRIMARY GRAFT DYSFUNCTION AFTER LUNG TRANSPLANTATION

(71) Applicants: Marc de Perrot, Toronto (CA); Shaf Keshavjee, Toronto (CA)

(72) Inventors: Marc de Perrot, Toronto (CA); Shaf Keshavjee, Toronto (CA)

(73) Assignee: UNIVERSITY HEALTH NETWORK, Toronto, ON (CA)

(21) Appl. No.: 13/640,690

(22) PCT Filed: Apr. 12, 2011
(86) PCT No.: PCT/CA11/00375

§ 371 Date: Oct. 11, 2012

Related U.S. Application Data
(60) Provisional application No. 61/323,090, filed on Apr. 12, 2010.

(51) Int. Cl. C12Q 1/68; C40B 30/04; G06F 19/00; C40B 40/06

(52) U.S. Cl. 506/9; 435/6.1; 506/16; 435/6.11; 702/20

(57) ABSTRACT

A method for determining pulmonary fibrosis subtype and/or prognosis in a subject having pulmonary fibrosis comprising: a. determining an expression profile by measuring the gene expression levels of a plurality of genes selected from genes listed in Table 1, 2, 3, 4 7, 8, 9, and/or 10, in a sample from the subject; and b. classifying the subject as having a good prognosis or a poor prognosis based on the expression profile; wherein a good prognosis predicts decreased risk of post lung transplant primary graft dysfunction, and wherein a poor prognosis predicts an increased risk of post lung transplant primary graft dysfunction.

### RELATED APPLICATION

This is a Patent Cooperation Treaty Application which claims the benefit of 35 U.S.C. 119 based on the priority of corresponding U.S. Provisional Patent Application No. 61/323,090, filed Apr. 12, 2010, which is incorporated herein in its entirety.

### FIELD

The disclosure relates to methods and compositions for classifying subtypes of pulmonary fibrois, diagnosing pulmonary fibrosis subtypes in a subject and determining the risk of primary graft dysfunction in a lung transplant recipient.

### INTRODUCTION

Secondary Pulmonary Hypertension (PH) is a frequent complication of Pulmonary Fibrosis. PH has a significant (negative) prognostic impact. While the pathological features of Secondary PH in PF are similar to those of Primary PH, the correlation with Pulmonary Function Tests is poor. It is currently unknown whether Secondary PH in IPF is causative or consequential, and whether PF patients with Secondary PH represent a distinct phenotype of the disease.

Lung transplantation is often the only therapeutic option for patients with PF. The results of lung transplantation in PF are currently limited by the risk of primary graft dysfunction. Primary graft dysfunction occurs in up to 50% of patients with PF undergoing lung transplantation and is the main cause of postoperative death after lung transplantation. Risk factors for the development of primary graft dysfunction in PF are not well defined.

### SUMMARY

In an aspect, the disclosure includes a method for determining pulmonary fibrosis subtype and/or prognosis in a subject having pulmonary fibrosis comprising:
a. determining an expression profile by measuring the gene expression levels of a plurality of genes selected from genes listed in Table 1, 2, 3, 4 7, 8, 9, and/or 10, in a sample from the subject; and
b. classifying the subject as having a good prognosis or a poor prognosis based on the expression profile;
   wherein a good prognosis predicts decreased risk of post lung transplant primary graft dysfunction, and wherein a poor prognosis predicts an increased risk of post lung transplant primary graft dysfunction.

In an embodiment, the method comprises:
a) calculating a first measure of similarity between a first expression profile and a good prognosis reference profile and a second measure of similarity between the first expression profile and a poor prognosis reference profile; the first expression profile comprising the expression levels of a first plurality of genes in a sample of the subject; the good prognosis reference profile comprising, for each gene in the first plurality of genes, the average expression level of the gene in a plurality of good prognosis subjects; and the poor prognosis reference profile comprising, for each gene in the first plurality of genes, the average expression level of the gene in a plurality of poor prognosis subjects, the first plurality of genes comprising at least 5 of the genes listed in Table 1, 2, 3, 4 7, 8, 9, and/or 10; and
b) classifying the subject as having a good prognosis if the first expression profile has a higher similarity to the good prognosis reference profile than to the poor prognosis reference profile, or classifying the subject as poor prognosis if the first expression profile has a higher similarity to the poor prognosis reference profile than to the good prognosis reference profile.

Another aspect of the disclosure includes a computer-implemented method for determining a prognosis of a subject having PF comprising: classifying, on a computer, the subject as having a good prognosis or a poor prognosis based on an expression profile comprising measurements of expression levels of a plurality of genes in a sample from the subject, the plurality of genes, comprising at least 5 genes listed in Table 1, 2, 3, 4 7, 8, 9, and/or 10; wherein a good prognosis predicts a decreased risk of PGD post lung transplant, and wherein a poor prognosis predicts an increased risk of PGD post lung transplant.

A further aspect of the disclosure includes a computer system comprising:
a) a database including records comprising reference expression profiles associated with clinical outcomes, each reference profile comprising the expression levels of a plurality of genes listed in Table 1, 2, 3, 4 7, 8, 9, and/or 10;
b) a user interface capable of receiving and/or inputting a selection of gene expression levels of a plurality of genes, the plurality comprising at least 5 genes listed in Table 1, 2, 3, 4 7, 8, 9, and/or 10, for use in comparing to the gene reference expression profiles in the database;
c) an output that displays a prediction of clinical outcome according to the expression levels of the plurality of genes.

Yet a further aspect includes a composition or kit comprising a plurality of analyte specific reagents (ASRs), optionally probes or primers, for determining expression of a plurality of genes.

Another aspect of the disclosure includes an array comprising for each gene in a plurality of genes, the plurality of genes being at least 5 of the genes listed in Table 1, 2, 3, 4 7, 8, 9, and/or 10, one or more polynucleotide probes complementary and hybridizable to a coding sequence in the gene.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the disclosure will now be described in relation to the drawings in which:

FIG. 1: Impact of PH on Prognosis

FIG. 2: Schematic of Method

FIG. 3: Signal Histogram

FIG. 4: Source of Variation

FIG. 5: SAM Analysis—Detection of Differentially Expressed Genes

FIG. 6: Levels of Gene Expression for Specific Genes

FIG. 7: Upregulated Gene Sets in PH Group

FIG. 8: No Title

FIG. 9: Clustering/Class Prediction Analysis

FIG. 10: Cluster analysis

FIG. 11: Intermediate group (mPAP 21-39 mmHg)—45 patients

FIG. 12: Cluster analysis

FIG. 13: All groups—84 Patients

FIG. 14: Cluster analysis

FIG. 15: RT-PCR analysis of Gene Expression

### DESCRIPTION OF VARIOUS EMBODIMENTS

### I. Definitions

As used herein “an expression profile” refers to, for a plurality of genes, gene expression levels and/or pattern of gene expression levels that is, for example, useful for class prediction for example for diagnosing pulmonary fibrosis (PF) subtype and/or for predicting risk of primary graft dysfunction (PGD). For example, an expression profile can comprise the expression levels of at least 5 or more genes listed in Table 1, 2, 3, 4 7, 8, 9, and/or 10 and the gene expression levels can be compared to one or more reference profiles, and based on similarity to a reference profile known to be associated with particular classes, be diagnostically or prognostically predicted to belong to a certain class. For example, the expression profile can include the expression of at least 5 genes associated with the PH group and/or at least 5 genes in no PH group.

A “reference expression profile” or “reference profile” as used herein refers to the expression signature (e.g. gene expression levels and/or pattern) of a plurality of genes or a gene, associated with a PF subtype and/or risk of PGD in a PF patient. The reference expression profile is identified using one or more samples comprising lung cells, for example lung tissue biopsies, wherein the expression is similar between related samples defining an outcome class and is different to unrelated samples defining a different outcome class such that the reference expression profile is associated with a particular class or clinical outcome. The reference expression profile is accordingly a reference profile or reference signature of the expression of 5 or more genes listed in Table 1, 2, 3, 4 7, 8, 9, and/or 10 to which the expression levels of the corresponding genes in a patient sample are compared in methods for determining or predicting clinical subtype and/or outcome, e.g. good prognosis (e.g. decreased risk of PGD) or poor prognosis (e.g. increased risk of PGD). A reference expression profile associated with good prognosis can be referred to a good prognosis reference profile and a reference expression profile associated with a poor prognosis can be referred to as a poor prognosis reference profile.

As used herein, the term “pulmonary hypertension gene expression profile” or “PH profile” refers to a pattern of gene expression that is seen in subjects with pulmonary hypertension PF (e.g. and a subset of intermediate PF) and includes for example increased expression of 5 or more genes listed in Table 1 or Table 3 or Table 7.

As used herein the term “no pulmonary hypertension gene expression profile” or “no-PH profile” or non-PH profile refers to the pattern of gene expression that is seen in subjects with no pulmonary hypertension PF and a subset of intermediate PF and includes for example increased expression of 5 or more genes listed in Table 2 or Table 4 or Table 9.

As used herein, the term “pulmonary arterial pressure” or “PAP” means the direct measurement of the pulmonary pressures through for example, a pulmonary artery catheter advanced into the pulmonary artery.

This is the most accurate way to obtain measurement of the pulmonary pressures and the mean pulmonary artery is the number used to diagnosed PH and defined the severity of PH.

As used herein, the term “outcome” or “clinical outcome” refers to the resulting course of disease and/or disease progression related to for example PF subtype and/or the clinical course of disease post transplant. For example, the outcome post transplant is determined based on assessment of for example PGD development and short or long term survival.

As used herein, “pulmonary fibrosis” or “PF” means is a chronic disease involving swelling and scarring of the alveoli (air sacs) and interstitial tissues of the lungs and the abnormal formation of fibre-like scar tissue in the lungs. PF can be caused secondary to certain diseases, but in the majority of cases the cause is unknown (e.g. idiopathic pulmonary fibrosis). Pulmonary fibrosis is a spectrum disorder that includes mild forms and severe disease. Other names for PF include for example, “Interstitial pulmonary fibrosis”, fibrosing alveolitis”, “intersititial pneumonitis” and “Hamman-Rich syndrome”.

As used herein “PF subtype” means a group within the spectrum of pulmonary fibrosis disease that can be distinguished on the basis of expression profile, for example, having expression similar to a pulmonary hypertension gene expression profile and/or a no pulmonary hypertension gene expression profile.

As used herein, “ISHLT criteria” refers to the definition of primary graft dysfunction established by the International Society for Heart and Lung Transplantation. ISHLT criteria defines three groups of primary graft dysfunction according to the gas exchange and chest x-ray findings.

As used herein, the term “primary graft dysfunction” or “PGD” in relation to a lung graft means acute lung injury developing postoperatively in a lung transplant recipient. The diagnosis can for example, be based on the gas exchange (PaO2/FiO2 ratio) and presence of infiltrates on the chest x-ray. Primary graft dysfunction is divided into three groups according to the severity of the dysfunction as mild (PGD-I) with a PaO2/FiO2 ratio of more than 300 and infiltrates on chest-x-ray, moderate (PGD-II) with a PaO2/FiO2 ratio between 200 and 300 and infiltrates on chest x-ray, and severe (PGD-III) with PaO2/FiO2 ratio of less than 200 and infiltrates on chest x-ray. Other terms used for PGD in the literature include for example, reperfusion edema, pulmonary edema, ischemia-reperfusion injury, and graft dysfunction.

As used herein, the term “risk of primary graft dysfunction (PGD)” means the likelihood of developing PGD.

As used herein “prognosis” refers to an indication of the likelihood of a particular clinical outcome, for example, an indication of the likelihood of PGD development, and/or likelihood of survival, and includes a “good prognosis” and a “poor prognosis”.

As used herein, “good prognosis” means a probable course of disease or disease outcome that has reduced morbidity and/or reduced mortality compared to the average for the disease or condition. For example, when referring to a lung transplant recipient, a good prognosis indicates that the subject is expected (e.g. predicted) to survive and/or have no, or low risk of PGD within a set time period, for example 30 days post transplant; and/or when referring to a PF subtype, a subject wherein the disease is not expected to progress or progress quickly e.g. a mild form of PF.

As used herein, “poor prognosis” means a probable course of disease or disease outcome that has increased morbidity and/or increased mortality compared to the average for the disease or condition. For example, when referring to a lung transplant recipient, a poor prognosis indicates that the subject is expected (e.g. predicted) to not survive and/or have high risk of PGD within a set time period, for example 30 days post transplant; and/or when referring to a PF subtype, a subject wherein the disease is expected to progress or progress quickly e.g. a severe form of PF. Severe forms of PF are expected to progress within for example, 6 to 12 months.

As used herein “gene set” refers to a plurality of genes whose expression is useful for predicting clinical outcome in a PF subject and includes for example, at least 5 genes, for example 6, 7, 8, 9, 10 or more genes listed in Table 1, 2, 3, 4 7, 8, 9, and/or 10. Gene set expression includes nucleic acids (including gene, pre-mRNA, and mRNA), polypeptides, as well as polymorphic variants, alleles and mutants. Truncated and alternatively spliced forms as well as complementary sequences are also included in the definition. Exemplary accession numbers for gene set genes are provided in Table 1 or 2 and are herein specifically incorporated by reference.

The term “expression level” of a gene as used herein refers to the measurable quantity of gene product produced by the gene in a sample of the subject e.g. patient, wherein the gene product can be a transcriptional product or a translational product. Accordingly, the expression level can pertain to a nucleic acid gene product such as mRNA or cDNA or a polypeptide gene product. The expression level is derived from a patient sample and/or a reference sample or samples, which can for example be detected de novo or correspond to a previous determination (e.g. pre-existing reference profile). The expression level can be determined or measured, for example, using microarray methods, PCR methods, and/or antibody based methods, as is known to a person of skill in the art.

The term “increased expression” and/or “increased level” as used herein refers to an increase in a level, or quantity, of a gene product (e.g. mRNA, cDNA or protein) in a sample that is measurable, compared to a control and/or reference sample. The term can also refer to an increase in the measurable expression, level of a given gene marker in a sample as compared with the measurable expression, level of a gene marker in a population of samples. For example, an expression level is altered if the ratio of the level in a sample as compared with a control or reference is greater than 1.0. For example, a ratio of greater than 1, 1.2, 1.5, 1.7, 2, 3, 4, 5, 6, 7, 8, 9, 10, 12, 15, 20 or more, or for example, 20%, 50%, 70%, 100%, 200%, 400%, 900% or more, compared to a reference sample or samples. Herein, for example, the genes were considered significant if a ratio greater than 1.5 was present. In terms of a profile “increased expression” means for each gene or a subset of genes assessed, the polypeptide or nucleic acid gene expression product is transcribed or translated at a detectably increased level. For example, as the expression and detection of gene expression can include noise, it would not be expected that each patient would have 100% of the signature. Accordingly, increases in for example at least 50% of the genes in the gene set would be expected to be predictive.

The term “decreased expressed” and/or “decreased level” as used herein means a polypeptide or nucleic acid gene expression product that is transcribed or translated at a detectably decreased level, in comparison to a reference sample or sample, for example in a sample comprising tissue from a fibrotic lung compared to a reference sample or samples associated with a particular prognosis. The term includes underexpression due to transcription, post-transcriptional processing, translation, post-translational processing, and/or protein and/or RNA stability. Underexpression can be 20%, 50%, 70%, 100%, 200%, 400%, 900% or more decreased, compared to a reference sample.

The term “hierarchical clustering” refers to a method of cluster analysis which seeks to build a hierarchy of clusters.

As used herein “sample” refers to any patient sample, including but not limited to a fluid, cell or tissue sample that comprises lung cells, which can be assayed for gene expression levels, particularly genes differentially expressed in patients having or not having PF (e.g. Table 1, 2, 3, 4 7, 8, 9, and/or 10 genes). The sample includes for example a lung biopsy, resected tissue, a frozen tissue sample, a fresh tissue specimen, a cell sample, and/or a paraffin embedded section or material.

The term “subject” also referred to as “patient” as used herein refers to any member of the animal kingdom, preferably a human being.

The term “hybridize” as used herein refers to the sequence specific non-covalent binding interaction with a complementary nucleic acid. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed. With respect to a chip array, appropriate stringency conditions are known in the art. For example, cleaned total RNA is used to generate double-stranded cDNA by reverse transcription, using a Superscript, double-stranded cDNA synthesis kit and an oligo deoxythymidylic acid primer with a T7 RNA polymerase promoter site added to the 3′ end. After second-strand synthesis, cDNA is cleaned with a GeneChip Sample Cleanup Module. Biotin-labeled cRNA is produced by in vitro transcription, using the Enzo BioArray high-yield RNA transcript labeling kit (Enzo Diagnostics, Farmingdale, N.Y.). Labeled cRNA is cleaned with a GeneChip Sample Cleanup Module, dried down and resuspended. Concentrated cRNA product is fragmented by metal-induced hydrolysis and the efficiency of the fragmentation procedure is checked by analyzing the size of the fragmented cRNA. Each fragmented sample is then used to prepare the hybridization cocktail. The hybridization cocktail can contain for example 100 mmol/L MES, 1 mol/L NaCl, 20 mmol/L ethylenediamine tetraacetic acid, 0.01% Tween 20, 0.1 mg/ml herring sperm DNA, 0.5 mg/ml acetylated bovine serum albumin, 50 pmol/L control oligonucleotide B2, 100 pmol/L eukaryotic hybridization controls, and 6 μg of fragmented sample. Samples are then hybridized to human genome arrays such as Affymetrix for 16 hours.

The term “stringent hybridization conditions” refers to conditions under which a probe will hybridize to its target subsequence, typically in a complex mixture of nucleic acids, but to no other sequences or only to sequences with greater than 95%, 96%, 97%, 98%, or 99% sequence identity. Stringent conditions are for example sequence-dependent and will be different in different circumstances. Longer sequences can require higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical, e.g. 95%, 95%, 97%, 98% or 99% identical. This occurs, for example, when a copy of a nucleic acid is created using the maximum codon degeneracy permitted by the genetic code. In such cases, the nucleic acids typically hybridize under moderately stringent hybridization conditions.

The term “microarray” as used herein refers to an ordered plurality of probes fixed to a solid surface that permits analysis such as gene analysis of a plurality of genes. A DNA microarray refers to an ordered plurality of DNA fragments fixed to a solid surface. For example, the microarray can be a gene chip. Methods of detecting gene expression and determining gene expression levels using arrays are well known in the art. Such methods are optionally automated.

The term “isolated nucleic acid sequence” as used herein refers to a nucleic acid substantially free of cellular material or culture medium when produced by recombinant DNA techniques, or chemical precursors, or other chemicals when chemically synthesized. The term “nucleic acid” is intended to include DNA and RNA and can be either double stranded or single stranded. The term nucleic acid is used interchangeably with gene, cDNA, mRNA, oligonucleotide and polynucleotide according to context.

The term “isolated polypeptide” or “isolated protein” used interchangeably as used herein refers to a polymer of amino acid residues.

The term “sequence identity” as used herein refers to the percentage of sequence identity between two or more polypeptide sequences or two or more nucleic acid sequences that have identity or a percent identity for example about 70% identity, 80% identity, 90% identity, 95% identity, 98% identity, 99% identity or higher identity or a specified region. To determine the percent identity of two or more amino acid sequences or of two or more nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). The percent identity between two sequences can be determined using techniques similar to those described above, with or without allowing gaps. In calculating percent identity, typically only exact matches are counted.

The term “analyte specific reagent” or “ASR” refers to any molecule including any chemical, nucleic acid sequence, polypeptide (e.g. receptor protein) or composite molecule and/or any composition that permits quantitative assessment of the analyte level. For example, the ASR can be for example a nucleic acid probe primer set, comprising a detectable label or aptamer that binds to, reacts with and/or responds to a gene in Table 1, 2, 3, 4 7, 8, 9, and/or 10. A gene specific ASR is herein referred to by reference to the gene, for example a “CLCA2” refers to an ASR such as a probe that specifically binds to a CLCA2 gene product in a manner to permit quantitation of the CLCA2 gene product (e.g. mRNA or corresponding of cDNA).

The term “specifically binds” as used herein refers to a binding reaction that is determinative of the presence of the analyte (e.g. polypeptide or nucleic acid) often in a heterogeneous population of macromolecules. For example, when the ASR is a probe, specifically binds refers to the specified probe under hybridization conditions binds to a particular gene sequence at least 1.5, at least 2 or at least 3 times background.

The term “probe” as used herein refers to a nucleic acid sequence that comprises a sequence of nucleotides that will hybridize specifically to a target nucleic acid sequence e.g. a gene listed in Table 1, 2, 3, 4 7, 8, 9, and/or 10. For example the probe comprises at least 10 or more bases or nucleotides that are complementary and hybridize contiguous bases and/or nucleotides in the target nucleic acid sequence. The length of probe depends on the hybridization conditions and the sequences of the probe and nucleic acid target sequence and can for example be 10-20, 21-70, 71-100, 101-500 or more bases or nucleotides in length. The probes can optionally be fixed to a solid support such as an array chip or a microarray chip.

The term “primer” as used herein refers to a nucleic acid sequence, whether occurring naturally as in a purified restriction digest or produced synthetically, which is capable of acting as a point of synthesis of when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques.

To produce human monoclonal antibodies, antibody producing cells (lymphocytes) can be harvested from a human having cancer and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with cancer cells and the monoclonal antibodies can be isolated.

Specific antibodies, or antibody fragments, reactive against particular target polypeptide gene product antigens (e.g. Table 1 or 2 polypeptide), can also be generated by screening expression libraries encoding immunoglobulin genes, or portions thereof, expressed in bacteria with cell surface components. For example, complete Fab fragments, VH regions and FV regions can be expressed in bacteria using phage expression libraries (See for example Ward et al., Nature 341:544-546 (1989); Huse et al., Science 246:1275-1281 (1989); and McCafferty et al., Nature 348:552-554 (1990)). A “detectable label” as used herein means an agent or composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “therapy” or “treatment” as used herein, refers to an approach aimed at obtaining beneficial or desired results, including clinical results and includes medical procedures and applications including for example surgery, pharmacological interventions, delivery of extra amount of oxygen through nasal cannulas and naturopathic interventions as well as test treatments. The phrase “PF therapy or treatment” refers to any approach including for example surgery, preventive interventions, prophylactic interventions and test treatments aimed at alleviating or ameliorating one or more symptoms, diminishing the extent of, stabilizing, preventing the spread of, delaying or slowing the progression of, ameliorating or palliating PF, or a subtype thereof, and/or associated symptoms and/or any associated complications thereof.

The term a “therapeutically effective amount”, “effective amount” or a “sufficient amount” of a compound of the present disclosure is a quantity sufficient to, when administered to a cell or a subject, including a mammal, for example a human, effect beneficial or desired results, including clinical results, and, as such, an “effective amount” or synonym thereto depends upon the context in which it is being applied. For example, in the context of PF, therapeutically effective amounts are used to treat, modulate, attenuate, reverse, or affect PF progression in a subject. For example, an “effective amount” is intended to mean that amount of a compound that is sufficient to treat, prevent or inhibit PF or a disease associated with PF. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art. Also, as used herein, a “therapeutically effective amount” of a compound is an amount which prevents, inhibits, suppresses or reduces PF (e.g., as determined by clinical symptoms in a subject as compared to a reference or comparison population. As defined herein, a therapeutically effective amount of a compound may be readily determined by one of ordinary skill by routine methods known in the art.

As used herein “a user interface device” or “user interface” refers to a hardware component or system of components that allows an individual to interact with a computer e.g. input data, or other electronic information system, and includes without limitation command line interfaces and graphical user interfaces.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The definitions and embodiments described in particular sections are intended to be applicable to other embodiments herein described for which they are suitable as would be understood by a person skilled in the art.

### II. Methods and Computer Products

Using gene expression profiling, distinct gene signatures were seen in subjects with pulmonary fibrosis depending on whether they had secondary pulmonary hypertension (PH group) or did not exhibit hypertension (NoPH group). Two distinct gene signatures were observed in PH and NoPH groups. PH patients showed an increased expression of genes, gene sets and networks related with myofibroblast proliferation, vascular remodeling, disruption of the basal membrane including Osteopontin, MMPI, MMP7, MMP13, Bone Morphogenic Protein Receptor 1 b, Fibroblast Growth Factor 14 and TP63. In contrast, NoPH patients showed a strong expression of genes involved in the inflammatory response, cell-mediated immune response and antigen presentation, including IL-6, PTX3, S100A8, VEGF, Endothelin Receptor B and Chemokine Ligand 10. Further, subjects with a No-PH-related gene signature were more likely to develop primary graft dysfunction (PGD) post-transplant compared to subjects with a PH-related gene signature. This suggests that distinct subtypes of PF exist that can be categorized based on gene signatures. These signatures are useful for identifying patients that belong to particular PF subtype for tailoring clinical management both prior to any or post lung transplant, stratifying patients in a clinical trial as well as for determining risk of PGD post transplant.

### A. Classification, Diagnostic and Therapeutic Methods

The present disclosure provides methods for determining PH subtype and/or providing a prognosis for PF subjects including for example post transplant by examining protein or RNA expression of markers listed in Table 1, 2, 3, 4 7, 8, 9, and/or 10, or a combination thereof in a sample from a subject.

Sets of genes, and corresponding expression levels in lung tissue from PF subjects associated with the presence or absence of severe secondary hypertension, which are predictive of clinical outcome (e.g. risk of PGD) post transplant are described herein.

It is demonstrated herein that subjects with PF and severe secondary hypertension exhibit increased expression of genes listed in Tables 1, 3, 7 and 8; and that subjects with PF and no secondary hypertension exhibit increased expression of genes listed in Tables 2, 4, 9 and 10. These signatures are useful for example, for predicting PF subtype and post-lung transplant outcome in subjects who have mild hypertension (e.g. mean pulmonary arterial pressure (mPAP) of for example 21-39 mmHg).
a. Accordingly in an aspect, the disclosure includes a method of classifying a subject with pulmonary fibrosis comprising: determining a gene expression level of a plurality of genes, comprising at least 1 for example 5 genes, selected from Table 1, 2, 3, 4 7, 8, 9, and/or 10 in a sample taken from the subject; and
b. classifying the subject as having a PH subtype when the expression levels of the plurality of genes is most similar to a PH profile and classifying the subject as a noPH subtype when the expression levels of the plurality of genes is most similar to a noPH profile.

In an embodiment, an increased expression of 5 or more genes in Table 7 classifies the subject has a PH subtype and/or an increased expression of 5 or more genes from Table 9 classifies the subject as a noPH subtype.

In an embodiment, the methods are used to classify a subject that has mild hypertension (e.g. mPAP (21-39 mmHg).

In an embodiment, the subject is classified for clinical management. In another embodiment, the subject is classified for stratifying patients in a clinical trial. In yet another embodiment, the subject is classified for predicting and managing the subject post lung transplant.

Accordingly, in another aspect, the disclosure includes a method for determining prognosis in a subject having PF, comprising:
a. determining a gene expression level of a plurality of genes, comprising at least 1 for example 5 genes, selected from Table 1, 2, 3, 4 7, 8, 9, and/or 10 in a sample taken from the subject; and
b. correlating the gene expression levels of the plurality of genes with a disease outcome prognosis.

In an embodiment, the method comprises:
a. determining an expression profile by measuring the gene expression levels of a plurality of genes, comprising at least 5 genes, selected from a Table 1 or 3, in a sample from the subject; and
b. classifying the subject as having a good prognosis or a poor prognosis based on the expression profile;
   wherein increased expression of the 5 or more genes is indicative that the subject is a noPH subtype and has a poor prognosis post lung transplant.

In another embodiment, the method comprises:
a. determining an expression profile by measuring the gene expression levels of a plurality of genes, comprising at least 5 genes, selected from a Table 2 or 4, in a sample from the subject; and
b. classifying the subject as having a good prognosis or a poor prognosis based on the expression profile;
   wherein increased expression of the 5 or more genes is indicative that the subject is a PH subtype and has a good prognosis post lung transplant.

Determination of prognosis, e.g. good prognosis or poor prognosis, or PF subtype can involve classifying a subject with PF based on the similarity of a subject's gene expression profile to one or more reference expression profile associated with a particular outcome and/or subtype, for example, by calculating a similarity to a reference expression profile associated with a good outcome post lung transplant (e.g. PH related signature) and/or a reference expression profile associated with a poor outcome post lung transplant (e.g. a noPH related signature). Accordingly, in an embodiment, the disclosure provides a method for classifying a subject having PF as having a good prognosis or a poor prognosis, comprising:
a. calculating a first measure of similarity between a first expression profile and a good prognosis reference profile and a second measure of similarity between the first expression profile and a poor prognosis reference profile; the first expression profile comprising the expression levels of a first plurality of genes in a sample of the subject; the good prognosis reference profile comprising, for each gene in the first plurality of genes, the average expression level of the gene in a plurality of good prognosis subjects; and the poor prognosis reference profile comprising, for each gene in the first plurality of genes, the average expression level of the gene in a plurality of poor prognosis subjects, the first plurality of genes comprising at least 5 of the genes listed in Table 1, 2, 3, 4 7, 8, 9, and/or 10; and
b. classifying the subject as having a good prognosis if the first expression profile has a higher similarity to the good prognosis reference profile than to the poor prognosis reference profile, or classifying the subject as poor prognosis if the first expression profile has a higher similarity to the poor prognosis reference profile than to the good prognosis reference profile.

Similarly, in an embodiment, the disclosure provides a method for classifying a subject's subtype of PF, comprising:
a. calculating a first measure of similarity between a first expression profile and a PF PH subtype reference profile and a second measure of similarity between the first expression profile and a PF noPH subtype reference profile; the first expression profile comprising the expression levels of a first plurality of genes in a sample of the subject; the PF PH subtype reference profile comprising, for each gene in the first plurality of genes, the average expression level of the gene in a plurality of PF PH subtype subjects; and the PF noPH subtype reference profile comprising, for each gene in the first plurality of genes, the average expression level of the gene in a plurality of PF noPH subtype subjects, the first plurality of genes comprising at least 5 of the genes listed in Table 1, 2, 3, 4 7, 8, 9, and/or 10; and
b. classifying the subject as having a PF PH subtype if the first expression profile has a higher similarity to the PF PH subtype reference profile than to the PF noPH subtype reference profile, or classifying the subject as PF noPH subtype if the first expression profile has a higher similarity to the PF noPH subtype reference profile than to the PF PH subtype reference profile.

Accordingly, in another embodiment, the method for classifying a subject having PF as having a PH subtype or noPH subtype; and/or a good prognosis or a poor prognosis, comprises:
a. calculating a measure of similarity between an expression profile and one or more subtype and/or prognosis reference profiles, the expression profile comprising the expression levels of a first plurality of genes in a sample taken from the subject; the one or more subtype and/or prognosis reference profiles comprising, for each gene in the plurality of genes, the average expression level of the gene in a plurality of subjects associated with the subtype and/or prognosis reference profile, for example a good prognosis reference profile and/or poor prognosis reference profile; the plurality of genes comprising at least 5 of the genes listed in Table 1, 2, 3, 4 7, 8, 9, and/or 10; and
b. classifying the subject as having the PH subtype and/or a good prognosis if the expression profile has a high similarity to the PH subtype and/or the good prognosis reference profile or has a higher similarity to the PH subtype and/or the good prognosis reference profile than to the PH poor prognosis reference profile or classifying the subject as having the noPH subtype and/or poor prognosis if the expression profile has a low similarity to the PH subtype and/or the good prognosis reference profile or has a higher similarity to the noPH subtype and/or the poor prognosis reference profile than to the PH subtype and/or good prognosis reference profile; wherein the expression profile has a high similarity to the PH subtype and/or the good prognosis reference profile if the similarity to the PH subtype and/or the good prognosis reference profile is above a predetermined threshold, or has a low similarity to the PH subtype and/or the good prognosis reference profile if the similarity to the PH subtype and/or good prognosis reference profile is below the predetermined threshold.

In addition, the expression levels of individual genes described herein may be individually prognostic. Accordingly, in an embodiment, the disclosure includes a method for identifying PF subtype comprising:
a. determining a gene expression level of at least 1 gene selected from Table 1, 3, 7, and/or 8, in a sample taken from the subject; and
b. classifying the subject as a PH subtype if the at least one gene is upregulated.

In another embodiment, the disclosure includes a method for identifying PF subtype comprising:
a. determining a gene expression level of at least 1 gene selected from Table 2, 4, 9, and/or 10, in a sample taken from the subject; and
b. classifying the subject as a non-PH subtype if the at least one gene is upregulated.

For example, it has been found that PTX3 by RT-PCR analysis is high in the non-PH group and not expressed at all in the PH group. Accordingly, in an embodiment the at least one gene comprises PTX3. In another embodiment, the at least one gene comprises CLCA2.

The methods described herein can be computer implemented. In an embodiment, the method further comprises: (c) displaying or outputting to a user interface device, a computer readable storage medium, or a local or remote computer system; the classification produced by the classifying step (b). In another embodiment, the method comprises displaying or outputting a result of one of the steps to a user interface device, a computer readable storage medium, a monitor, or a computer that is part of a network.

In another embodiment, the method comprises a computer-implemented method for determining a prognosis of a subject having PF comprising: classifying, on a computer, the subject as having a good prognosis or a poor prognosis based on an expression profile comprising measurements of expression levels of a plurality of genes in a sample from the subject, the plurality of genes, comprising at least 5 genes listed in Table 1, 2, 3, 4 7, 8, 9, and/or 10; wherein a good prognosis predicts a decreased risk of PGD post lung transplant, and wherein a poor prognosis predicts an increased risk of PGD post lung transplant.

The reference profiles can be pre-generated, for example the expression profiles can be comprised in a database or generated de novo. In an embodiment, the method comprises the steps of:
a. generating a good prognosis reference profile;
b. generating a poor prognosis reference profile;
c. generating a first expression profile of a subject with PH;
d. calculating a measure of similarly between the first expression profile and one or more of good prognosis reference profiles; and
e. classifying the subject as having a good prognosis if the first expression profile is similar, or has higher similarity, to the good prognosis reference profile and/or classifying the subject as having a poor prognosis if the first expression profile is similar, or has a higher similarity to the poor prognosis reference profile.

In another embodiment, the method comprises the steps of:
a. generating a PH subtype profile reference profile;
b. generating a no PH reference profile;
c. generating a first expression profile of a subject with PH;
d. calculating a measure of similarly between the first expression profile and one or more of the PH subtype reference profile; and
e. classifying the subject as having a PH subtype if the first expression profile is similar, or has higher similarity, to the PH subtype reference profile and/or classifying the subject as having a noPH subtype if the first expression profile is similar, or has a higher similarity to the noPH subtype reference profile.

In another embodiment the method comprises:
a. generating a good prognosis and/or PH subtype reference profile by hybridization of nucleic acids derived from the plurality of subjects having PH subtype PF against nucleic acids derived from a pool of samples from a plurality of subjects having PF;
b. generating a poor prognosis reference profile by hybridization of nucleic acids derived from the plurality of subjects having noPH subtype PF against nucleic acids derived from the pool of samples from the plurality of subjects;
c. generating a first expression profile by hybridizing nucleic acids derived from the sample taken from the subject against nucleic acids derived from the pool of samples from the plurality of subjects; and
d. calculating a first measure of similarity between the first expression profile and the PH subtype PF and/or good prognosis reference profile and the second measure of similarity between the first expression profile and the noPH subtype PF and/or poor prognosis reference profile, wherein if the first expression profile is more similar to the PH subtype PF and/or good prognosis reference profile than to the noPH subtype PF and/or poor prognosis reference profile, the subject is classified as having a PH subtype PF and/or good prognosis respectively, and if the first expression profile is more similar to the noPH subtype PF and/or poor prognosis reference profile than to the PH subtype PF and/or good prognosis reference profile, the subject is classified as having a noPH subtype PF and/or poor prognosis respectively.

In an embodiment, the good prognosis profile is generated by determining an average expression level for at least five genes selected from Table 1, 2, 3, 4 7, 8, 9, and/or 10 in a plurality of subjects having a good clinical outcome for example having a PH subtype of PF.

In an embodiment, the gene set or plurality of genes comprises at least 5 genes selected from Table 1, 2, 3, 4 7, 8, 9, and/or 10. In another embodiment, the gene set or plurality of genes comprises at least 6, 7, 8, 9, 10, 11, 12, 13, 14 or 15 genes listed in Table 1, 2, 3, 4 7, 8, 9, and/or 10. In another embodiment, the gene set or plurality of genes comprises 16-25, 26-35, 36-45, 46-55, 56-65, 66-75, 76-85, 86-95, 96-105, 106-115, 116-125, 126-135, 136-145, 146-155, 156-165, 166-175, 176-185, 186-195, 196-205, 206-215, 216-225, 226-233 genes listed in Table 1 and/or 2. In yet another embodiment, the gene set or plurality of genes comprises all the genes listed in Table 1. In another embodiment, the gene set or plurality of genes comprises all of the genes listed in Table 2. In a further embodiment, the gene set or plurality of genes, comprises 6-10, 11-15, 16-20 or more genes listed in Tables 3 and/or 4. In a further embodiment, the gene set or plurality of genes comprises the genes listed in Table 3 or the genes listed in Table 4. In yet a further embodiment, the gene set or plurality of genes consists of the genes listed in Table 1, 2, 3, 4 7, 8, 9, and/or 10, or a subset thereof.

In an embodiment, the fold change in a gene expression level is 1.5, 1.7, 2, 3, 4, 5, 6, 7, 8, 9, 10, 12, 15, 20 or more fold change compared to the expression of the corresponding gene of a reference profile or at least a 50%, 70%, 90%, 95%, 100%, 200%, 400%, 900%, or more increased or decreased, compared to a reference sample or profile.

A person skilled in the art would understand that not all the genes in a particular signature may be increased or decreased according to the reference profile. This may be due to for example noise in the detection of gene expression of these genes. Accordingly, in an embodiment, 70%, 80%, 85%, 90%, 95% of the genes profiled in a gene set exhibit increased expression level.

In another embodiment, the method for determining post transplant prognosis in a subject having PF, comprises:
a. determining an expression profile by measuring the gene expression levels of a plurality of genes selected from the genes listed in Table 1, 2, 3, 4 7, 8, 9, and/or 10, in a sample from the subject; and
b. classifying the subject as having a good prognosis or a poor prognosis based on the expression profile;
   wherein a good prognosis predicts decreased risk of PGD post lung transplant, and wherein a poor prognosis predicts an increased risk of PGD post lung transplant.

The classification is for example carried out by comparing the expression profile of the plurality of genes and comparing to a reference profile.

The described predictors are able to stratify patients according to clinical outcome. Accordingly the methods described herein can be used for example to select subjects for a clinical trial. So far, all studies to assess treatment impact on the outcome of PF have been negative. In the future, the ability to stratify patients according to their risk may improve the chances of success of future trials by using more appropriate therapy and better patients' selection. Accordingly, in an embodiment, the subject is a participant in a clinical trial to assess a candidate drug. n an embodiment the method further comprise using the subject's PF subtype information to select a subject population for a clinical trial. In another embodiment, the method further comprises using the subject's PF subtype information to stratify a subject population in a clinical trial. In another embodiment, the method further comprises using the subject's PF subtype information to stratify subjects that respond to a treatment from those who do not respond to a treatment, or subjects that have negative side effects from those who do not have negative side effects.

Also included in an embodiment, is a method of selecting a human subject for inclusion or exclusion in a clinical trial, the method comprising: classifying a subject as a PF PH subtype or a PF noPH subtype according to a method described herein comprising detecting the expression level of a plurality of genes and/or determining an expression profile; and including or excluding the subject if the expression level and/or profile indicates that the subject has a PF PH subtype or a PF noPH subtype. In an embodiment, the clinical trial is of a treatment for PF with secondary hypertension. In an embodiment, the clinical trial is of a treatment for PF without secondary hypertension.

Accurate classification can reduce the number of patients identified as high risk. Further, accurate classification allows for treatments to be tailored and for aggressive therapies with greater risks or side effects to be reserved for patients with poor outcome. Accordingly in another aspect, the disclosure includes a method further comprising the step of providing a PF and/or a PGD treatment regimen for a subject consistent with the disease outcome prognosis.

In another aspect, the disclosure includes a method of selecting or optimizing a PF or PDG treatment comprising:
a. determining a subject gene expression profile and prognosis according to a method described herein; and
b. selecting a treatment indicated by their prognosis.

For example, for subjects with poor prognosis, suitable treatments can include anti-inflammatory drugs, such as steroids or cyclophosphamide.

In an embodiment, the expression profile and/or treatment selected is transmitted to a caregiver of the subject. In another embodiment, the expression profile and/or treatment is transmitted over a network.

In yet another aspect, the disclosure provides a method of treating a subject with PF, the method comprising:
a. determining a subject gene expression profile and prognosis according to a method described herein;
b. treating the subject with a treatment indicated by their prognosis.

In an embodiment, the treatment is for PF. In another embodiment, the treatment is post lung transplant. In another embodiment, the treatment is for PGD. In an embodiment, the method comprises administering to a subject an effective therapeutic amount of a PF or PGD treatment indicated by the subject's expression profile.

In yet another embodiment, a method described herein also comprises first obtaining a sample from the subject. The sample, in an embodiment, comprises or is a lung biopsy or a surgical resection. In an embodiment, the sample comprises fresh tissue, frozen tissue sample, a cell sample, or a paraffin embedded sample. In an embodiment, the sample is submerged in a RNA preservation solution, for example to allow for storage.

In an embodiment, the sample is submerged in Trizol®. Frozen tissue is for example, maintained in liquid nitrogen until RNA can be processed. For RNA preparation, tissue can be homogenized in 5M guanidine isothiocyanate and purified using commercially-available RNA purification columns (e.g. Qiagen, Invitrogen) according to manufacturer's instructions. RNA is stored for example, at −80 C until use.

The sample in an embodiment is processed, for example, to obtain an isolated RNA fraction and/or an isolated polypeptide fraction. For example, the sample can be treated with a lysis solution e.g. to lyse the cells, to allow a detection agent access to the RNA species. The sample can also or alternatively be processed using a RNA isolation kit such as RNeasy to isolate RNA or a fraction thereof (e.g. mRNA). The sample is in an embodiment, treated with a RNAse inhibitor to prevent RNA degradation.

Wherein the gene expression level being determined is a nucleic acid, the gene expression levels can be determined using a number of methods for example hybridization to a probe or a microarray chip (e.g. an oligonucleotide array) or using primers and PCR amplification based methods, optionally multiplex PCR or high throughput sequencing. These methods are known in the art. For example a person skilled in the art would be familiar with the necessary normalizations necessary for each technique. For example, the expression measurements generated using multiplex PCR should be normalized by comparing the expression of the genes being measure to so called “housekeeping” genes, the expression of which should be constant over all samples, thus providing a baseline expression to compare against.

Accordingly, in an embodiment, determining the expression profile comprises contacting a sample comprising RNA or cDNA corresponding to the RNA (e.g. a processed sample from the subject) with an analyte specific reagent (ASR), for example an ASR that specifically binds and/or amplifies a nucleic acid product of a gene listed in Table 1, 2, 3, 4 7, 8, 9, and/or 10 such as CLCA2, for each gene of the plurality of genes and determining the expression level for each gene. For example, where the ASR specifically binds a nucleic acid expression product, a complex is formed between the ASR and target expression product. The expression level of each gene is thus determined by measuring complexes formed to determine the expression level of the gene. Also for example, where the ASR specifically and quantitatively amplifies a nucleic acid expression product, measuring the amount of the amplification product determines the level of gene expression. Thus contacting for example with a CLCA2 ASR, and measuring the complexes formed or the amplification product amounts is used to determine the expression level of the marker (i.e. CLCA2) in the sample. Similarly contacting with a IRF1 ASR is used to determine the expression level of the IRF1 marker. In an embodiment, the step of correlating the gene expression levels and/or classifying the subject comprises determining whether or not the expression profile, for example whether the RNA representing 5 or more of the genes listed in Table 1, 2, 3, 4 7, 8, 9, and/or 10 4, is altered in the sample when compared to corresponding RNA expression levels representing each marker nucleic acid of a comparison population of subjects, for example a PH subtype PF class or a noPH subtype PF class.

In an embodiment, the ASR is a nucleic acid molecule (e.g. an oligonucleotide). In an embodiment, the nucleic acid molecule comprises probe. In another embodiment, the ASR comprises a primer set that amplifies a Table 1, 2, 3, 4 7, 8, 9, and/or 10 nucleic acid gene product (e.g. RNA and/or corresponding cDNA). In another embodiment, the nucleic acid molecule is comprised in an array.

The expression level can also be the polypeptide expression level. A person skilled in the art will appreciate that a number of methods can be used to determine the amount of a polypeptide product of a gene described herein, including immunoassays such as Western blots, ELISA, and immunoprecipitation followed by SDS-PAGE, as well as immunocytochemistry or immunohistochemistry.

Accordingly, in an embodiment, determining the expression profile comprises contacting a sample comprising polypeptide (e.g. a processed sample from the subject) with an analyte specific reagent (ASR), for example an ASR that specifically binds a polypeptide product of a gene listed in Table 1, 2, 3, 4 7, 8, 9, and/or 10 such as CLCA2, for each gene of the plurality of genes and determining the expression level for each gene. For example, where the ASR specifically binds a polypeptide expression product, a complex is formed between the ASR and target product. The expression level of each gene is thus determined by measuring complexes formed to determine the expression level of the gene. Thus contacting for example with a CLCA2 ASR, and measuring the complexes formed is used to determine the expression level of the marker (i.e. CDLCA2) in the sample. In an embodiment, the step of correlating the gene expression levels and/or classifying the subject comprises determining whether or not the expression profile, for example whether the polypeptide level representing 5 or more of the genes listed in Table 1, 2, 3, 4 7, 8, 9, and/or 10, is altered in the sample when compared to corresponding polypeptide levels representing each marker polypeptide of a comparison population of subjects, for example a PH subtype PF class or a noPH subtype PF class.

In an embodiment, the ASR is an antibody. In an embodiment, the antibody is a monoclonal antibody. In a further embodiment, the antibody is comprised in an array.

### B. Computer Product

Another aspect of the disclosure includes a computer product for implementing the methods described herein e.g. for predicting prognosis, selecting patients for a clinical trial, or selecting therapy. Accordingly in an embodiment, the computer product is a non-transitory computer readable storage medium with an executable program stored thereon, wherein the program is for predicting outcome in a subject having PF, and wherein the program instructs a microprocessor to perform the steps of any of the methods described herein.

A further aspect includes a computer system comprising:
a. a database including records comprising reference expression profiles associated with clinical outcomes, each reference profile comprising the expression levels of a plurality of genes listed in Table 1, 2, 3, 4 7, 8, 9, and/or 10;
b. a user interface capable of receiving and/or inputting a selection of gene expression levels of a plurality of genes, the plurality comprising at least 5 genes listed in Table 1, 2, 3, 4 7, 8, 9, and/or 10, for use in comparing to the gene reference expression profiles in the database;
c. an output that displays a prediction of clinical outcome according to the expression levels of the plurality of genes.

In an embodiment, the computer system is used to carry out the methods described herein.

### B. Novel Candidate Therapeutics

A further aspect of the disclosure includes a method of identifying agents for use in the treatment of PF. Clinical trials seek to test the efficacy of new therapeutics. The efficacy is often only determinable after many months of treatment. The methods disclosed herein are useful for monitoring the expression of genes associated with prognosis. Accordingly, changes in gene expression levels which are associated with a better prognosis are indicative the agent is a candidate as a chemotherapeutic.

Accordingly in an embodiment, the disclosure provides a method for identifying candidate agents for use in treatment of PF and/or PGD comprising:
a. obtaining an expression level for at least 5 genes listed in Table 1, 2, 3, 4 7, 8, 9, and/or 10 in a first test sample of a lung cell or a population of cells comprising lung cells, wherein the cell or population of cells is optionally in vitro or in vivo;
b. contacting for example, by incubating, the cell or population of cells with a test agent;
c. obtaining an expression level for the at least 5 genes in a second test sample, wherein the second test sample is obtained subsequent to incubating the cell culture with the test agent;
d. comparing the expression level of the at least 5 genes in the first and second test samples to a good prognosis reference expression profile and a poor prognosis reference expression profile of the at least 5 genes;
   wherein a change in the expression level of the genes in the second sample indicating a greater similarity to a good prognosis reference profile indicates that the agent is a candidate therapeutic.

The test samples are in an embodiment a population of cells in culture, wherein the first test sample is obtained prior to incubating the population with a test agent and the second sample is from the same culture of cells and obtained subsequent to contact with the test agent. In another embodiment, the cell or population of cells is in vivo, wherein the first test sample is obtained before administering a test agent to an animal comprising PF and/or PGD and the second test sample is taken from the same or similar location subsequent to administering the test agent. A person skilled in the art will be familiar with various animal models, cell culture techniques and cell lines that are useful for the methods described herein.

### III. Compositions, Arrays and Kits

An aspect provides a composition comprising a plurality of probes or primers for determining expression of a plurality of genes. In an embodiment, the plurality comprises and/or consists of at least 5 genes.

Another aspect of the disclosure includes an array comprising for each gene in a plurality of genes, the plurality of genes being at least 5 of the genes listed in Table 1, 2, 3, and/or 4 one or more polynucleotide probes complementary and hybridizable to a coding sequence in the gene. In an embodiment, the gene set or the plurality of genes comprises at least 6, 7, 8, 9, 10, 11, 12, 13, 14 or 15 genes listed in Table 1, 2, 3, 4 7, 8, 9, and/or 10. In another embodiment, the plurality of genes comprises 16-25, 26-35, 36-45, 46-55, 56-65, 66-75, 76-85, 86-95, 96-105, 106-115, 116-125, 126-135, 136-145, 146-155, 156-165, 166-175, 176-185, 186-195, 196-205, 206-215, 216-225, 226-233 genes listed in Table 1 and/or 2. In yet another embodiment, the plurality of genes comprising all the genes listed in Table 1, 2, 3, 4 7, 8, 9, and/or 10. In yet a further embodiment, the plurality of genes consists of the genes listed in Table 1, 2, 3, 4 7, 8, 9, and/or 10, or a subset thereof.

The array can be a microarray, a DNA array and/or a tissue array. In an embodiment, the array is a multi-plex qRT-PCR-based array.

Another aspect includes a kit for determining prognosis in a subject having PF comprising:
a. an array described herein;
b. one or more or specimen collector and RNA preservation solution; and optionally
c. instructions for use.

In an embodiment, the specimen collector comprises a sterile vial or tube suitable for receiving a biopsy or other sample. In an embodiment, the specimen collector comprises RNA preservation solution. In another embodiment, RNA preservation solution is added subsequent to the reception of sample.

In an embodiment the RNA preservation solution comprises one or more inhibitors of RNAse. In another embodiment, the RNA preservation solution comprises Trizol®.

Another aspect includes a kit for determining prognosis in a subject having PF comprising:
d. a plurality of probes comprising at least two probes, wherein each probe hybridizes and/or is complementary to a nucleic acid sequence corresponding to a gene selected from Table 1, 2, 3, 4 7, 8, 9, and/or 10; and optionally
e. one or more of specimen collector, RNA preservation solution and instructions for use.

In an embodiment, the kit comprises at least 2, at least 5, at least 10 or at least 15 probes. In another embodiment, the kit comprises a plurality of probes, for at least 5 genes listed in Table 1, 2, 3, 4 7, 8, 9, and/or 10 (e.g. for detecting gene expression of at least 5 genes). For example, one or more probes can be directed to the detection of gene expression of one gene. In an embodiment, the kit comprises probes for 16-25, 26-35, 36-45, 46-55, 56-65, 66-75, 76-85, 86-95, 96-105, 106-115, 116-125, 126-135, 136-145, 146-155, 156-165, 166-175, 176-185, 186-195, 196-205, 206-215, 216-225, 226-233 genes listed in Tables 1 and/or 2. In an embodiment, the kit comprises 16-25, 26-35, 36-45, 46-55, 56-65, 66-75, 76-85, 86-95, 96-105, 106-115, 116-125, 126-135, 136-145, 146-155, 156-165, 166-175, 176-185, 186-195, 196-205, 206-215, 216-225, 226-233 probes. In another embodiment, the plurality of probes comprises and/or consists of at least one probe for each gene in Table 1, 2, 3, 4 7, 8, 9, and/or 10.

Another aspect of the disclosure is a kit for determining prognosis in a subject having PF comprising:
a. a plurality of antibodies comprising at least two antibodies, wherein each antibody of the set is specific for a polypeptide corresponding to a gene selected from Table 1; and optionally
b. one or more of specimen collector, polypeptide preservation solution and instructions for use.

In an embodiment, the kit comprises a plurality of antibodies specific for polypeptides corresponding to at least 2, 3, 4, 5, 6, 7, 8, 9 or at least 10 of the genes listed in Table 1, 2, 3, 4 7, 8, 9, and/or 10. In another embodiment, the kit comprises a plurality of antibodies specific for polypeptides corresponding to at least 16-25, 26-35, 36-45, 46-55, 56-65, 66-75, 76-85, 86-95, 96-105, 106-115, 116-125, 126-135, 136-145, 146-155, 156-165, 166-175, 176-185, 186-195, 196-205, 206-215, 216-225, 226-233 of the genes listed in Table 1 and/or 2. In yet another embodiment, the kit comprises a plurality of antibodies specific for polypeptides corresponding to the genes listed in Table 1, 2, 3, 4 7, 8, 9, and/or 10.

In an embodiment, the antibody or probe is labeled. The label is preferably capable of producing, either directly or indirectly, a detectable signal. For example, the label may be radio-opaque or a radioisotope, such as ^{'}H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

In another embodiment, the detectable signal is detectable indirectly. A person skilled in the art will appreciate that a number of methods can be used to determine the amount of a polypeptide product of a gene described herein, including immunoassays such as Western blots, ELISA, and immunoprecipitation followed by SDS-PAGE, as well as immunocytochemistry or immunohistochemistry. The kit can accordingly in certain embodiments comprise reagents for one or more of these methods, for example molecular weight markers, standards or analyte controls.

The kit can comprise in an embodiment, one or more probes or one or more antibodies specific for a gene. In another embodiment, the set or probes or antibodies comprise probes or antibodies wherein each probe or antibody detects a different gene listed in Table 1, 2, 3, 4 7, 8, 9, and/or 10.

In an embodiment, the kit is used for a method described herein.

The following non-limiting examples are illustrative of the present disclosure:

### EXAMPLES

### Example 1

### Methods

116 lung tissues biopsies were obtained from the recipient organs of PF patients undergoing a Lung Transplant (LTx). PAP was measured intraoperatively before starting LTx. The mean PAP was calculated according to the following formula: DPAP+⅓(SPAP-DPAP).

For the development analysis, RNA was extracted from explanted lungs in 84 patients with PF (52 males, age 59±8 years, BMI 26±4, mPAP 29±12 mmHg, 69 bilateral LTx). 17 patients had severe Pulmonary Hypertension (PH) (mean PAP 40 mmHg; PH Group), 22 had no PH (mPAP 20 mmHg; NoPH Group), and 45 had intermediate mPAP (21-39 mmHg; Intermediate Group).

RNA was extracted from 32 more patients (19 males, age 55±13 years, BMI 27±5, mPAP 31±18 mmHg, 19 bilateral LTx) for the validation analysis.

RNA was isolated with TRizol® Reagent (Invitrogen, Cat. No. 15596-018); a clean up step was performed then with RNeasy MinElute Cleanup kit (QIAGEN, Cat. No. 74204). Totally 50 μl RNA was collected for each sample and divided to two part, 10 μl and 40 μl. 10 μl is for RNA qualification and microarray; 40 μl is for subsequent assay.

cDNA was synthesized in 80 μl from 4 μg of RNA with High-Capacity cDNA Reverse Transcription kits (ABI, Cat No. 4374966). cDNA-synthesis was carried out on a PTC-100′ Programmable Thermal controller (MJ research Inc. USA), at 25° C. for 10 min, 37° C. for 120 min, 85° C. for 5 min, 4° C. for ∞.

RNA was qualified by RNA nano chips on an Agilent 2100 Bilanalyzer (Agilent Technologies, USA) and Microarray was performed by Genechip® Human Gene 1.0 ST on an Affymetrix Genechip Scanner 3000 and Genechip® Fluidics Station 450 (JMP, USA).

Microarray analysis included SAM analysis (detection of differentially expressed genes in different groups), Ingenuity Pathway analysis (Pathways/Networks Discovery Analysis) and Gene Set Enrichment Analysis.

### Results

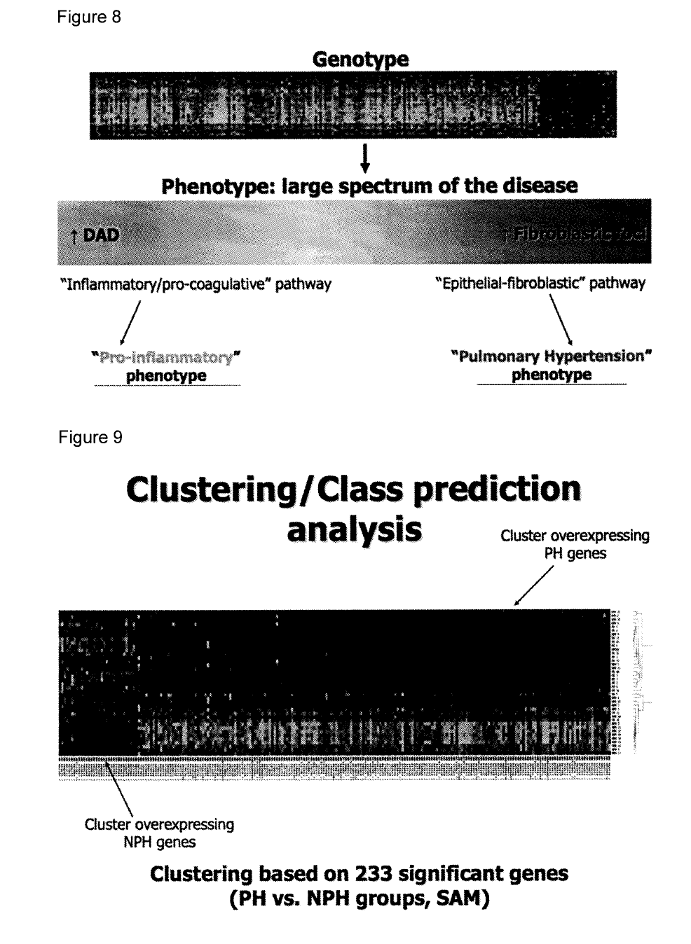

Two distinct gene signatures were observed in PH and NoPH groups (FIG. 8). PH patients showed an increased expression of genes, gene sets and networks related with myofibroblast proliferation, vascular remodeling, disruption of the basal membrane, including Osteopontin, MMP1, MMP7, MMP13, Bone Morphogenic Protein Receptor 1b, Fibroblast Growth Factor 14 and TP63. In contrast, NoPH patients showed a strong expression of genes involved in the inflammatory response, cell-mediated immune response and antigen presentation, including IL-6, PTX3, S100A8, and Chemokine Ligand 10.

In the Intermediate group, two-dimensional hierarchical clustering based on 233 differentially expressed genes (PH vs. NoPH group) dichotomized subjects into two distinct subgroups.

The impact of different gene signatures on Primary Graft Dysfunction (PGD) after LTx was next analyzed. PGD on arrival in the ICU was defined according to the ISHLT criteria.

In the Intermediate group, patients clustered in the subgroup with increased expression of NoPH-related genes had higher incidence of PGD II-III (52% vs. 14%, p=0.006).

Looking at the whole population, PAP did not predict PGD. However, the NoPH-related gene signature was associated with a higher incidence of PGD II-III when compared to the PH-related gene signature (40% vs. 17%, p=0.022). A logistic regression model in the whole population showed that clustering algorithm based on PH vs. NoPH gene signature was the only significant predictor of PGD (Chi square 5.6, p=0.017), while PAP and type of operation were not.

The gene expression signatures based on 233 differentially expressed genes (PH vs. NoPH group) were analyzed in a validation cohort of 32 patients. Once again, two-dimensional hierarchical clustering dichotomized subjects into two distinct subgroups, and again the NoPH-related gene signature was associated with a higher incidence of PGD II-III (36%) when compared to the PH-related gene signature (21%). Further results are provided in Example 2.

### Conclusion

Although PAP is not a predictor of PGD, PF patients exhibit two distinct gene expression profiles that are predictive of risk of PGD post-LTx. Gene expression profiles based on PAP may identify distinct phenotypes of Pulmonary Fibrosis, with different clinical courses, different pathological and radiographic features and different outcomes after Lung Transplantation.

TABLE 1
Genes upregulated in PH group

| Gene ID | Gene Name | Fold Change |
|---|---|---|
| NM_033197 // C20orf114 // chromosome 20 open | 8061894 | 3.446499274 |
| reading frame 114 // 20q11.21 // 92 | | |
| NM_002443 // MSMB // microseminoprotein, beta- // | 7927529 | 2.505881155 |
| 10q11.2 // 4477 /// NM_138634 | | |
| NM_024889 // C10orf81 // chromosome 10 open | 7930593 | 2.421552037 |
| reading frame 81 // 10q25.3 // 79949 | | |
| NM_006536 // CLCA2 // CLCA family member 2, | 7902702 | 2.403953757 |
| chloride channel regulator // 1p31-p | | |
| NM_024687 // ZBBX // zinc finger, B-box domain | 8091887 | 2.358667372 |
| containing // 3q26.1 // 79740 /// | | |
| NM_000424 // KRT5 // keratin 5 // 12q12-q13 // 3852 | 7963427 | 2.356155243 |
| /// ENST00000252242 // KRT5 | | |
| NM_031422 // CHST9 // carbohydrate (N- | 8022666 | 2.324397258 |
| acetylgalactosamine 4-0) sulfotransferase | | |
| ENST00000295941 // ASB14 // ankyrin repeat and | 8088315 | 2.292064761 |
| SOCS box-containing 14 // 3p21.1 | | |
| BC101698 // CXorf59 // chromosome X open reading | 8166690 | 2.278205289 |
| frame 59 // Xp21.1 // 286464 | | |
| NM_024423 // DSC3 // desmocollin 3 // 18q12.1 // | 8022692 | 2.246758781 |
| 1825 /// NM_001941 // DSC3 // d | | |
| ENST00000351747 // DNHD2 // dynein heavy chain | 8088299 | 2.238079292 |
| domain 2 // 3p14.3 // 201625 | | |
| NM_198564 // DNAH12L // dynein, axonemal, heavy | 8088322 | 2.185200718 |
| chain 12-like // 3p14.3 // 37534 | | |
| NM_006017 // PROM1 // prominin 1 // 4p15.32 // 8842 | 8099476 | 2.169090892 |
| /// ENST00000265014 // PROM1 | | |
| NM_031457 // MS4A8B // membrane-spanning 4- | 7940323 | 2.161465826 |
| domains, subfamily A, member 8B // 11 | | |
| BC093659 // C13orf30 // chromosome 13 open | 7968866 | 2.134474711 |
| reading frame 30 // 13q14.11 // 14480 | | |
| NM_024593 // EFCAB1 // EF-hand calcium binding | 8150691 | 2.108812737 |
| domain 1 // 8q11.21 // 79645 /// | | |
| NM_002421 // MMP1 // matrix metallopeptidase 1 | 7951271 | 2.107128487 |
| (interstitial collagenase) // 11q | | |
| NM_025145 // C10orf79 // chromosome 10 open | 7936201 | 2.101495261 |
| reading frame 79 // 10q25.1 // 80217 | | |
| NM_006919 // SERPINB3 // serpin peptidase inhibitor, | 8023696 | 2.06986812 |
| clade B (ovalbumin), member | | |
| NM_012443 // SPAG6 // sperm associated antigen 6 // | 7926622 | 2.066891447 |
| 10p12.2 // 9576 /// NM_17224 | | |
| NM_152632 // CXorf22 // chromosome X open reading | 8166671 | 2.058872387 |
| frame 22 // Xp21.1 // 170063 / | | |
| NM_001080537 // S100A1L // Protein S100-A1-like // | 8080863 | 2.045585857 |
| 3p14.2 // 132203 /// ENST0000 | | |
| NM_206996 // SPAG17 // sperm associated antigen | 7918973 | 2.0322758 |
| 17 // 1p12 // 200162 /// ENST000 | | |
| NM_006269 // RP1 // retinitis pigmentosa 1 | 8146468 | 2.028563855 |
| (autosomal dominant) // 8q11-q13 // 6 | | |
| NM_024694 // C6orf103 // chromosome 6 open | 8122561 | 2.019809305 |
| reading frame 103 // 6q24.3 // 79747 | | |
| NM_001004303 // C1orf168 // chromosome 1 open | 7916506 | 1.980415072 |
| reading frame 168 // 1p32.2 // 199 | | |
| AK304339 // FAM154B // family with sequence | 7985398 | 1.975124117 |
| similarity 154, member B // 15q25.2 | | |
| BC015442 // LOC200383 // similar to Dynein heavy | 8043059 | 1.973795706 |
| chain at 16F // 2p11.2 // 20038 | | |
| NM_003357 // SCGB1A1 // secretoglobin, family 1A, | 7940654 | 1.970013525 |
| member 1 (uteroglobin) // 11q1 | | |
| XM_001726086 // TMEM212 // transmembrane | 8083897 | 1.963956453 |
| protein 212 // 3q26.31 // 100130245 | | |
| NM_173081 // ARMC3 // armadillo repeat containing 3 | 7926638 | 1.958674585 |
| // 10p12.31 // 219681 /// EN | | |
| NM_005727 // TSPAN1 // tetraspanin 1 // 1p34.1 // | 7901175 | 1.95201924 |
| 10103 /// ENST00000372003 // T | | |
| NM_025063 // C1orf129 // chromosome 1 open | 7907232 | 1.944898392 |
| reading frame 129 // 1q24.3 // 80133 | | |
| NM_001040058 // SPP1 // secreted phosphoprotein 1 | 8096301 | 1.944602013 |
| // 4q21-q25 // 6696 /// NM_000 | | |
| NM_173565 // RSPH10B // radial spoke head 10 | 8138009 | 1.929939955 |
| homolog B (Chlamydomonas) // 7p22.1 | | |
| NM_001372 // DNAH9 // dynein, axonemal, heavy | 8004957 | 1.928268585 |
| chain 9 // 17p12 // 1770 /// NM_00 | | |
| NM_173565 // RSPH10B // radial spoke head 10 | 8131452 | 1.917769181 |
| homolog B (Chlamydomonas) // 7p22.1 | | |
| NM_018272 // CASC1 // cancer susceptibility | 7961844 | 1.917088731 |
| candidate 1 // 12p12.1 // 55259 /// | | |
| NM_176884 // TAS2R43 // taste receptor, type 2, | 7961295 | 1.914757103 |
| member 43 // 12p13.2 // 259289 / | | |
| NM_000096 // CP // ceruloplasmin (ferroxidase) // | 8091385 | 1.91016002 |
| 3q23-q25 // 1356 /// ENST00000 | | |
| NM_002458 // MUC5B // mucin 5B, oligomeric | 7937612 | 1.908920727 |
| mucus/gel-forming // 11p15.5 // 72789 | | |
| NM_178827 // IQUB // IQ motif and ubiquitin domain | 8142646 | 1.901803207 |
| containing // 7q31.32 // 1548 | | |
| NM_017539 // DNAH3 // dynein, axonemal, heavy | 8000034 | 1.894945475 |
| chain 3 // 16p12.2 // 55567 /// EN | | |
| NM_080860 // RSPH1 // radial spoke head 1 homolog | 8070603 | 1.894470119 |
| (Chlamydomonas) // 21q22.3 // | | |
| ENST00000389394 // DNAH6 // dynein, axonemal, | 8043071 | 1.88935965 |
| heavy chain 6 //—// 1768 /// E | | |
| NM_025052 // YSK4 // yeast Sps1/Ste20-related | 8055361 | 1.888226517 |
| kinase 4 (S. cerevisiae) // 2q21.3 | | |
| NM_145010 // C10orf63 // chromosome 10 open | 7932598 | 1.86584846 |
| reading frame 63 // 10p12.1 // 21967 | | |
| BC111738 // FLJ23834 // hypothetical protein | 8135341 | 1.86542469 |
| FLJ23834 // 7q22.2 // 222256 /// BC | | |
| NM_144980 // C6orf118 // chromosome 6 open | 8130664 | 1.864996282 |
| reading frame 118 // 6q27 // 168090 / | | |
| NM_145286 // STOML3 // stomatin (EPB72)-like 3 // | 7971126 | 1.857119577 |
| 13q13.3 // 161003 /// ENST0000 | | |
| BC073916 // C1orf173 // chromosome 1 open reading | 7917019 | 1.846950927 |
| frame 173 // 1p31.1 // 127254 | | |
| NM_005143 // HP // haptoglobin // 16q22.1 // 3240 /// | 7997188 | 1.844662996 |
| NM_001126102 // HP // hapt | | |
| NM_032165 // LRRIQ1 // leucine-rich repeats and IQ | 7957433 | 1.840389797 |
| motif containing 1 // 12q21.3 | | |
| NM_032229 // SLITRK6 // SLIT and NTRK-like family, | 7972239 | 1.839158514 |
| member 6 // 13q31.1 // 84189 | | |
| NM_178456 // C20orf85 // chromosome 20 open | 8063601 | 1.835018081 |
| reading frame 85 // 20q13.32 // 1286 | | |
| NM_018076 // ARMC4 // armadillo repeat containing 4 | 7932744 | 1.832849349 |
| // 10p12.1-p11.23 // 55130 / | | |
| NM_178135 // HSD17B13 // hydroxysteroid (17-beta) | 8101637 | 1.830396574 |
| dehydrogenase 13 // 4q22.1 // | | |
| NM_024690 // MUC16 // mucin 16, cell surface | 8033674 | 1.829136754 |
| associated // 19p13.2 // 94025 /// | | |
| NM_012397 // SERPINB13 // serpin peptidase | 8021603 | 1.826013807 |
| inhibitor, clade B (ovalbumin), membe | | |
| NM_004363 // CEACAM5 // carcinoembryonic | 8029086 | 1.822472416 |
| antigen-related cell adhesion molecule | | |
| NM_001013626 // LRRC67 // leucine rich repeat | 8151127 | 1.820995596 |
| containing 67 // 8q13.1-q13.2 // 2 | | |
| NM_173645 // DNHL1 // dynein heavy chain-like 1 // | 8043043 | 1.817839549 |
| 2p11.2 // 284944 /// BC 104884 | | |
| NM_207437 // DNAH10 // dynein, axonemal, heavy | 7959681 | 1.817100594 |
| chain 10 // 12q24.31 // 196385 // | | |
| NM_178452 // LRRC50 // leucine rich repeat | 7997556 | 1.814032623 |
| containing 50 // 16q24.1 // 123872 // | | |
| AK304357 // FLJ16686 // FLJ16686 protein // 4p14 // | 8094533 | 1.807665754 |
| 401124 /// BC157885 // FLJ16 | | |
| NM_181807 // DCDC1 // doublecortin domain | 7947322 | 1.806764672 |
| containing 1 // 11p13 // 341019 /// EN | | |
| NM_002851 // PTPRZ1 // protein tyrosine | 8135774 | 1.802886209 |
| phosphatase, receptor-type, Z polypeptid | | |
| NM_002652 // PIP // prolactin-induced protein // 7q34 | 8136839 | 1.794744067 |
| // 5304 /// ENST0000029100 | | |
| NM_032821 // HYDIN // hydrocephalus inducing | 8002446 | 1.782778953 |
| homolog (mouse) // 16q22.1-q22.3 // | | |
| NM_012144 // DNAI1 // dynein, axonemal, | 8154892 | 1.782174936 |
| intermediate chain 1 // 9p21-p13 // 2701 | | |
| NM_005554 // KRT6A // keratin 6A // 12q12-q13 // | 7963421 | 1.780758362 |
| 3853 /// ENST00000330722 // KRT | | |
| NM_001122961 // C1orf194 // chromosome 1 open | 7918294 | 1.780027948 |
| reading frame 194 // 1p13.3 // 127 | | |
| BC035083 // C6orf165 // chromosome 6 open reading | 8121015 | 1.7749185 |
| frame 165 // 6q15 // 154313 // | | |
| ENST00000330194 // C10orf107 // chromosome 10 | 7927723 | 1.769827391 |
| open reading frame 107 // 10q21.2 | | |
| NM_032821 // HYDIN // hydrocephalus inducing | 8002492 | 1.76648179 |
| homolog (mouse) // 16q22.1-q22.3 // | | |
| NM_001013625 // C1orf192 // chromosome 1 open | 7921862 | 1.761603024 |
| reading frame 192 // 1q23.3 // 257 | | |
| NM_018406 // MUC4 // mucin 4, cell surface | 8092978 | 1.759007268 |
| associated // 3q29 // 4585 /// NM_004 | | |
| NM_178550 // C1orf110 // chromosome 1 open | 7921909 | 1.758365942 |
| reading frame 110 // 1q23.3 // 339512 | | |
| NM_002275 // KRT15 // keratin 15 // 17q21.2 // 3866 | 8015337 | 1.751920359 |
| /// ENST00000254043 // KRT15 | | |
| NM_020775 // KIAA1324 // KIAA1324 // 1p13.3 // | 7903592 | 1.745264432 |
| 57535 /// ENST00000234923 // KIAA | | |
| NM_198520 // C12orf63 // chromosome 12 open | 7957688 | 1.743320252 |
| reading frame 63 // 12q23.1 // 37446 | | |
| NM_144992 // VWA3B // von Willebrand factor A | 8043747 | 1.738666757 |
| domain containing 3B // 2q11.2 // | | |
| NM_033413 // LRRC46 // leucine rich repeat | 8008040 | 1.737043235 |
| containing 46 // 17q21.32 // 90506 // | | |
| NM_001031741 // NEK10 // NIMA (never in mitosis | 8085867 | 1.734434229 |
| gene a)-related kinase 10 // 3p | | |
| NM_024626 // VTCN1 // V-set domain containing T | 7918936 | 1.733173098 |
| cell activation inhibitor 1 // 1 | | |
| NM_001944 // DSG3 // desmoglein 3 (pemphigus | 8020762 | 1.727508402 |
| vulgaris antigen) // 18q12.1-q12.2 | | |
| NM_001004330 // PLEKHG7 // pleckstrin homology | 7957514 | 1.725482945 |
| domain containing, family G (with | | |
| NM_199289 // NEK5 // NIMA (never in mitosis gene | 7971757 | 1.720328645 |
| a)-related kinase 5 // 13q14.3 | | |
| AJ132086 // DNAH6 // dynein, axonemal, heavy chain | 8043055 | 1.714163607 |
| 6 //—// 1768 /// U61736 / | | |
| NM_000673 // ADH7 // alcohol dehydrogenase 7 | 8101904 | 1.712959184 |
| (class IV), mu or sigma polypeptide | | |
| AK057222 // C2orf39 // chromosome 2 open reading | 8040672 | 1.712056699 |
| frame 39 // 2p23.3 // 92749 /// | | |
| BC105284 // LOC100130771 // EF-hand domain- | 8142079 | 1.71056711 |
| containing protein LOC100130771 // 7q | | |
| NM_001447 // FAT2 // FAT tumor suppressor homolog | 8115302 | 1.708610725 |
| 2 (Drosophila) // 5q32-q33 // | | |
| NM_198469 // MORN5 // MORN repeat containing 5 // | 8157632 | 1.706697412 |
| 9q33.2 // 254956 /// ENST00000 | | |
| NM_173086 // KRT6C // keratin 6C // 12q13.13 // | 7963410 | 1.703244863 |
| 286887 /// NM_005554 // KRT6A // | | |
| AK128035 // DCDC5 // doublecortin domain containing | 7947282 | 1.699451604 |
| 5 // 11p14.1-p13 // 196296 / | | |
| NM_144575 // CAPN13 // calpain 13 // 2p22-p21 // | 8051275 | 1.694581955 |
| 92291 /// ENST00000406764 // CA | | |
| NM_018897 // DNAH7 // dynein, axonemal, heavy | 8057821 | 1.691921736 |
| chain 7 // 2q32.3 // 56171 /// ENS | | |
| NM_199328 // CLDN8 // claudin 8 // 21q22.11 // 9073 | 8069795 | 1.690418805 |
| /// ENST00000399899 // CLDN8 | | |
| NM_001039845 // MDH1B // malate dehydrogenase | 8058462 | 1.68887342 |
| 1B, NAD (soluble) // 2q33.3 // 130 | | |
| NM_178824 // WDR49 // WD repeat domain 49 // | 8091922 | 1.683686992 |
| 3q26.1 // 151790 /// ENST0000030837 | | |
| NM_021827 // CCDC81 // coiled-coil domain | 7942941 | 1.683573724 |
| containing 81 // 11q14.2 // 60494 /// | | |
| NM_012128 // CLCA4 // chloride channel, calcium | 7902738 | 1.682161603 |
| activated, family member 4 // 1p | | |
| NM_144647 // CAPSL // calcyphosine-like // 5p13.2 // | 8111506 | 1.681723917 |
| 133690 /// NM_001042625 // | | |
| NM_138796 // SPATA17 // spermatogenesis | 7909768 | 1.679181505 |
| associated 17 // 1q41 // 128153 /// ENST | | |
| NM_025244 // TSGA10 // testis specific, 10 // 2q11.2 // | 8054166 | 1.669019831 |
| 80705 /// NM_182911 // T | | |
| NM_145020 // CCDC11 // coiled-coil domain | 8023314 | 1.666845794 |
| containing 11 // 18q21.1 // 220136 /// | | |
| AK125070 // FLJ43080 // hypothetical protein | 8113483 | 1.665716541 |
| LOC642987 // 5q22.1 // 642987 /// B | | |
| NM_002427 // MMP13 // matrix metallopeptidase 13 | 7951309 | 1.664005699 |
| (collagenase 3) // 11q22.3 // 4 | | |
| NM_152590 // IFLTD1 // intermediate filament tail | 7961875 | 1.662508278 |
| domain containing 1 // 12p12.1 | | |
| BC028708 // C20orf26 // chromosome 20 open | 8061272 | 1.657035755 |
| reading frame 26 // 20p11.23 // 26074 | | |
| NM_032821 // HYDIN // hydrocephalus inducing | 8002470 | 1.65408665 |
| homolog (mouse) // 16q22.1-q22.3 // | | |
| NM_207430 // C11orf88 // chromosome 11 open | 7943740 | 1.653313815 |
| reading frame 88 // 11q23.1 // 39994 | | |
| NM_031916 // ROPN1L // ropporin 1-like // 5p15.2 // | 8104492 | 1.652012128 |
| 83853 /// ENST00000274134 // | | |
| NM_001203 // BMPR1B // bone morphogenetic | 8096511 | 1.650840115 |
| protein receptor, type IB // 4q22-q24 | | |
| NM_032821 // HYDIN // hydrocephalus inducing | 8002481 | 1.646518738 |
| homolog (mouse) // 16q22.1-q22.3 // | | |
| NM_025087 // FLJ21511 // hypothetical protein | 8094988 | 1.644295508 |
| FLJ21511 // 4p12-p11 // 80157 /// | | |
| ENST00000298953 // C12orf55 // chromosome 12 | 7957673 | 1.639365771 |
| open reading frame 55 // 12q23.1 // | | |
| NM_152327 // AK7 // adenylate kinase 7 // 14q32.2 // | 7976578 | 1.637372102 |
| 122481 /// ENST00000267584 | | |
| NM_001010892 // RSHL3 // radial spokehead-like 3 // | 8121622 | 1.632704454 |
| 6q22.1 // 345895 /// ENST000 | | |
| NM_032554 // GPR81 // G protein-coupled receptor | 7967325 | 1.627582102 |
| 81 // 12q24.31 // 27198 /// ENS | | |
| NM_023915 // GPR87 // G protein-coupled receptor | 8091515 | 1.62555709 |
| 87 // 3q24 // 53836 /// ENST000 | | |
| ENST00000406767 // RP1-199H16.1 // hypothetical | 8076113 | 1.625382272 |
| LOC388900 // 22q13.1 // 388900 | | |
| NM_002423 // MMP7 // matrix metallopeptidase 7 | 7951217 | 1.622091122 |
| (matrilysin, uterine) // 11q21-q2 | | |
| NM_003106 // SOX2 // SRY (sex determining region | 8084165 | 1.620000852 |
| Y)-box 2 // 3q26.3-q27 // 6657 | | |
| NM_145054 // WDR16 // WD repeat domain 16 // | 8004889 | 1.617692599 |
| 17p13.1 // 146845 /// NM_001080556 | | |
| — | 8088335 | 1.616796767 |
| NM_152709 // STOX1 // storkhead box 1 // 10q21.3 // | 7927915 | 1.613023234 |
| 219736 /// ENST00000298596 / | | |
| BC034296 // C4orf22 // chromosome 4 open reading | 8096061 | 1.611812474 |
| frame 22 // 4q21.21 // 255119 / | | |
| NM_001042524 // FRMPD2L1 // FERM and PDZ | 7933279 | 1.607066169 |
| domain containing 2 like 1 // 10q11.22 | | |
| NM_001042524 // FRMPD2L1 // FERM and PDZ | 7933394 | 1.607066169 |
| domain containing 2 like 1 // 10q11.22 | | |
| NM_003645 // SLC27A2 // solute carrier family 27 | 7983650 | 1.606455915 |
| (fatty acid transporter), membe | | |
| NM_053285 // TEKT1 // tektin 1 // 17p13.2 // 83659 /// | 8011990 | 1.606455707 |
| ENST00000338694 // TEKT1 | | |
| NM_000927 // ABCB1 // ATP-binding cassette, sub- | 8140782 | 1.606137197 |
| family B (MDR/TAP), member 1 // | | |
| NM_003722 // TP63 // tumor protein p63 // 3q28 // | 8084766 | 1.606034801 |
| 8626 /// NM_001114978 // TP63 | | |
| NM_152410 // PACRG // PARK2 co-regulated // 6q26 | 8123303 | 1.601244553 |
| // 135138 /// NM_001080378 // P | | |
| NM_031956 // TTC29 // tetratricopeptide repeat | 8103064 | 1.601226184 |
| domain 29 // 4q31.23 // 83894 /// | | |
| NM_024763 // WDR78 // WD repeat domain 78 // | 7916789 | 1.601226154 |
| 1p31.3 // 79819 /// NM_207014 // WD | | |
| NM_152548 // FAM81B // family with sequence | 8106950 | 1.601222415 |
| similarity 81, member B // 5q15 // 1 | | |
| NM_198524 // TEX9 // testis expressed 9 // 15q21.3 // | 7983828 | 1.600861832 |
| 374618 /// ENST00000352903 | | |
| NM_031294 // LRRC48 // leucine rich repeat | 8005289 | 1.592437752 |
| containing 48 // 17p11.2 // 83450 /// | | |
| NM_014157 // CCDC113 // coiled-coil domain | 7996198 | 1.592307102 |
| containing 113 // 16q21 // 29070 /// | | |
| NM_145740 // GSTA1 // glutathione S-transferase A1 | 8127072 | 1.589750248 |
| // 6p12.1 // 2938 /// ENST000 | | |
| NM_012101 // TRIM29 // tripartite motif-containing 29 | 7952290 | 1.589335722 |
| // 11q22-q23 // 23650 /// | | |
| NM_178821 // WDR69 // WD repeat domain 69 // | 8048870 | 1.588564317 |
| 2q36.3 // 164781 /// ENST0000030993 | | |
| NM_001115131 // C6 // complement component 6 // | 8111864 | 1.587207765 |
| 5p13 // 729 /// NM_000065 // C6 | | |
| BC118982 // LOC339809 // KIAA2012 protein // | 8047505 | 1.58656612 |
| 2q33.1 // 339809 /// ENST0000033180 | | |
| NM_001085447 // C2orf77 // chromosome 2 open | 8056710 | 1.586199578 |
| reading frame 77 // 2q31.1 // 12988 | | |
| BC027878 // C1orf87 // chromosome 1 open reading | 7916629 | 1.583482778 |
| frame 87 // 1p32.1 // 127795 // | | |
| NM_000463 // UGT1A1 // UDP | 8049349 | 1.582876111 |
| glucuronosyltransferase 1 family, polypeptide A1 // 2 | | |
| BC141809 // C9orf117 // chromosome 9 open reading | 8158081 | 1.579227112 |
| frame 117 // 9q34.11 // 286207 | | |
| NM_007072 // HHLA2 // HERV-H LTR-associating 2 // | 8081488 | 1.579216019 |
| 3q13.13 // 11148 /// ENST00000 | | |
| NM_019894 // TMPRSS4 // transmembrane protease, | 7944164 | 1.578166402 |
| serine 4 // 11q23.3 // 56649 /// | | |
| NM_144715 // EFHB // EF-hand domain family, | 8085732 | 1.577680247 |
| member B // 3p24.3 // 151651 /// ENS | | |
| NM_130387 // ASB14 // ankyrin repeat and SOCS | 8088292 | 1.57764282 |
| box-containing 14 // 3p21.1 // 142 | | |
| NM_020879 // CCDC146 // coiled-coil domain | 8133770 | 1.576911196 |
| containing 146 // 7q11.23 // 57639 // | | |
| NM_152498 // WDR65 // WD repeat domain 65 // | 7900639 | 1.575998107 |
| 1p34.2 // 149465 /// ENST0000029639 | | |
| NM_016571 // GLULD1 // glutamate-ammonia ligase | 8127380 | 1.575896436 |
| (glutamine synthetase) domain co | | |
| NM_203454 // APOBEC4 // apolipoprotein B mRNA | 7922804 | 1.575722023 |
| editing enzyme, catalytic polypept | | |
| BC047053 // C1orf141 // chromosome 1 open reading | 7916822 | 1.575011344 |
| frame 141 // 1p31.3 // 400757 | | |
| NM_145235 // FANK1 // fibronectin type III and | 7931281 | 1.574682346 |
| ankyrin repeat domains 1 // 10q26 | | |
| NM_181426 // CCDC39 // coiled-coil domain | 8092295 | 1.572191089 |
| containing 39 // 3q26.33 // 339829 /// | | |
| NM_020995 // HPR // haptoglobin-related protein // | 7997192 | 1.570395672 |
| 16q22.1 // 3250 /// ENST00000 | | |
| NM_201548 // CERKL // ceramide kinase-like // | 8057463 | 1.566760229 |
| 2q31.3 // 375298 /// NM_001030311 | | |
| — | 8134429 | 1.561744992 |
| NM_018004 // TMEM45A // transmembrane protein | 8081288 | 1.557398659 |
| 45A // 3q12.2 // 55076 /// ENST000 | | |
| NM_145172 // WDR63 // WD repeat domain 63 // | 7902660 | 1.555817898 |
| 1p22.3 // 126820 /// ENST0000029466 | | |
| NM_033364 // C3orf15 // chromosome 3 open reading | 8081903 | 1.55357475 |
| frame 15 // 3q12-q13.3 // 8987 | | |
| NM_006217 // SERPINI2 // serpin peptidase inhibitor, | 8091910 | 1.54461384 |
| clade I (pancpin), member 2 | | |
| NM_003777 // DNAH11 // dynein, axonemal, heavy | 8131719 | 1.541283036 |
| chain 11 // 7p21 // 8701 /// ENST | | |
| NM_004415 // DSP // desmoplakin // 6p24 // 1832 /// | 8116780 | 1.539978448 |
| NM_001008844 // DSP // desmo | | |
| NM_006952 // UPK1B // uroplakin 1B // 3q13.3-q21 // | 8081826 | 1.53904102 |
| 7348 /// ENST00000264234 // | | |
| NR_003561 // DPY19L2P2 // dpy-19-like 2 | 8141882 | 1.537283289 |
| pseudogene 2 (C. elegans) // 7q22.1 // 3 | | |
| NM_001018071 // FRMPD2 // FERM and PDZ domain | 7933446 | 1.537235938 |
| containing 2 // 10q11.22 // 143162 | | |
| — | 7972661 | 1.536949297 |
| NM_024867 // SPEF2 // sperm flagellar 2 // 5p13.2 // | 8104856 | 1.535885017 |
| 79925 /// NM_144722 // SPEF | | |
| NM_024783 // AGBL2 // ATP/GTP binding protein-like | 7947947 | 1.533710914 |
| 2 // 11p11.2 // 79841 /// ENS | | |
| NM_144668 // WDR66 // WD repeat domain 66 // | 7959330 | 1.531741971 |
| 12q24.31 // 144406 /// ENST00000288 | | |
| AK295603 // FLJ39061 // hypothetical protein | 8047492 | 1.531521835 |
| FLJ39061 // 2q33.1 // 165057 /// AK | | |
| NM_025257 // SLC44A4 // solute carrier family 44, | 8125149 | 1.531401491 |
| member 4 // 6p21.3 // 80736 // | | |
| NM_025257 // SLC44A4 // solute carrier family 44, | 8178653 | 1.531401491 |
| member 4 // 6p21.3 // 80736 // | | |
| NM_025257 // SLC44A4 // solute carrier family 44, | 8179861 | 1.531401491 |
| member 4 // 6p21.3 // 80736 // | | |
| NM_000564 // IL5RA // interleukin 5 receptor, alpha // | 8085062 | 1.527459234 |
| 3p26-p24 // 3568 /// NM_1 | | |
| — | 7924461 | 1.524612483 |
| NM_054023 // SCGB3A2 // secretoglobin, family 3A, | 8108995 | 1.52000069 |
| member 2 // 5q32 // 117156 /// | | |
| NM_130897 // DYNLRB2 // dynein, light chain, | 7997374 | 1.519290053 |
| roadblock-type 2 // 16q23.3 // 8365 | | |
| NM_145170 // TTC18 // tetratricopeptide repeat | 7934334 | 1.517463559 |
| domain 18 // 10q22.2 // 118491 // | | |
| NM_030906 // STK33 // serine/threonine kinase 33 // | 7946365 | 1.51692399 |
| 11p15.3 // 65975 /// ENST000 | | |
| NM_145650 // MUC15 // mucin 15, cell surface | 7947156 | 1.516750679 |
| associated // 11p14.3 // 143662 /// | | |
| — | 8100758 | 1.51293718 |
| NM_001062 // TCN1 // transcobalamin I (vitamin B12 | 7948444 | 1.5127043 |
| binding protein, R binder fam | | |
| NM_001080850 // RP4-692D3.1 // hypothetical protein | 7900555 | 1.511729764 |
| LOC728621 // 1p34.2 // 72862 | | |
| ENST00000354752 // ANKRD18B // ankyrin repeat | 8154823 | 1.508836405 |
| domain 18B // 9p13.3 // 441459 | | |
| NM_152701 // ABCA13 // ATP-binding cassette, sub- | 8132743 | 1.506502927 |
| family A (ABC1), member 13 // 7 | | |
| NM_173672 // PPIL6 // peptidylprolyl isomerase | 8128726 | 1.506371594 |
| (cyclophilin)-like 6 // 6q21 // 2 | | |
| NM_006194 // PAX9 // paired box 9 // 14q12-q13 // | 7973974 | 1.505821541 |
| 5083 /// ENST00000402703 // PA | | |
| NM_175929 // FGF14 // fibroblast growth factor 14 // | 7972650 | 1.504343872 |
| 13q34 // 2259 /// NM_004115 | | |
| NM_178499 // CCDC60 // coiled-coil domain | 7959108 | 1.504301327 |
| containing 60 // 12q24.23 // 160777 // | | |
| NM_144646 // IGJ // immunoglobulin J polypeptide, | 8100827 | 1.501090365 |
| linker protein for immunoglobu | | |

TABLE 2
Genes upregulated in no-PH group

| Gene ID | Gene Name | Fold Change |
|---|---|---|
| NM_014391 // ANKRD1 // ankyrin repeat domain 1 | 7934979 | 2.557437596 |
| (cardiac muscle) // 10q23.31 // 2 | | |
| NM_002164 // INDO // indoleamine-pyrrole 2,3 | 8146092 | 2.014066973 |
| dioxygenase // 8p12-p11 // 3620 /// | | |
| NM_001045 // SLC6A4 // solute carrier family 6 | 8013989 | 1.991849304 |
| (neurotransmitter transporter, se | | |
| NM_181789 // GLDN // gliomedin // 15q21.2 // 342035 | 7983704 | 1.948202348 |
| /// ENST00000335449 // GLDN | | |
| NM_002852 // PTX3 // pentraxin-related gene, rapidly | 8083594 | 1.928624132 |
| induced by IL-1 beta // 3q2 | | |
| NM_000600 // IL6 // interleukin 6 (interferon, beta 2) // | 8131803 | 1.905854162 |
| 7p21 // 3569 /// ENST0 | | |
| NM_001565 // CXCL10 // chemokine (C—X—C motif) | 8101126 | 1.874837194 |
| ligand 10 // 4q21 // 3627 /// ENS | | |
| NM_001872 // CPB2 // carboxypeptidase B2 (plasma) | 7971444 | 1.868512672 |
| // 13q14.11 // 1361 /// NM_016 | | |
| NM_006732 // FOSB // FBJ murine osteosarcoma viral | 8029693 | 1.782882539 |
| oncogene homolog B // 19q13.3 | | |
| NM_145913 // SLC5A8 // solute carrier family 5 (iodide | 7965769 | 1.764974451 |
| transporter), member 8 // | | |
| NM_002964 // S100A8 // S100 calcium binding protein | 7920244 | 1.730117571 |
| A8 // 1q21 // 6279 /// ENST0 | | |
| NM_003853 // IL18RAP // interleukin 18 receptor | 8044049 | 1.704319453 |
| accessory protein // 2q12 // 880 | | |
| NM_005409 // CXCL11 // chemokine (C—X—C motif) | 8101131 | 1.690944621 |
| ligand 11 // 4q21.2 // 6373 /// E | | |
| NM_002416 // CXCL9 // chemokine (C—X—C motif) | 8101118 | 1.651270804 |
| ligand 9 // 4q21 // 4283 /// ENST0 | | |
| NM_176870 // MT1M // metallothionein 1M // 16q13 // | 7995787 | 1.630074393 |
| 4499 /// ENST00000379818 // | | |
| — | 7965787 | 1.627842745 |
| NM_003955 // SOCS3 // suppressor of cytokine | 8018864 | 1.616964129 |
| signaling 3 // 17q25.3 // 9021 /// | | |
| NM_001945 // HBEGF // heparin-binding EGF-like | 8114572 | 1.614382312 |
| growth factor // 5q23 // 1839 /// | | |
| NM_014143 // CD274 // CD274 molecule // 9p24 // | 8154233 | 1.596683771 |
| 29126 /// ENST00000381577 // CD2 | | |
| NM_001462 // FPR2 // formyl peptide receptor 2 // | 8030860 | 1.593652949 |
| 19q13.3-q13.4 // 2358 /// NM_0 | | |
| — | 7999384 | 1.593023667 |
| NM_000602 // SERPINE1 // serpin peptidase inhibitor, | 8135069 | 1.591223894 |
| clade E (nexin, plasminogen | | |
| NM_005328 // HAS2 // hyaluronan synthase 2 // | 8152617 | 1.588156106 |
| 8q24.12 // 3037 /// ENST0000030392 | | |
| NM_005946 // MT1A // metallothionein 1A // 16q13 // | 7995806 | 1.58487013 |
| 4489 /// ENST00000290705 // | | |
| AK123303 // FLJ41309 // hypothetical protein | 8106727 | 1.565996008 |
| LOC645079 // 5q14.2 // 645079 /// A | | |
| NM_007231 // SLC6A14 // solute carrier family 6 | 8169504 | 1.564534562 |
| (amino acid transporter), member | | |
| NM_052941 // GBP4 // guanylate binding protein 4 // | 7917561 | 1.550285533 |
| 1p22.2 // 115361 /// ENST000 | | |
| NM_002198 // IRF1 // interferon regulatory factor 1 // | 8114010 | 1.545478842 |
| 5q31.1 // 3659 /// ENST00 | | |
| NM_002089 // CXCL2 // chemokine (C—X—C motif) | 8100994 | 1.531041649 |
| ligand 2 // 4q21 // 2920 /// ENST0 | | |
| NM_005621 // S100A12 // S100 calcium binding | 7920238 | 1.527410798 |
| protein A12 // 1q21 // 6283 /// ENS | | |
| NM_025243 // SLC19A3 // solute carrier family 19, | 8059538 | 1.524043736 |
| member 3 // 2q37 // 80704 /// | | |
| NM_014358 // CLEC4E // C-type lectin domain family | 7960900 | 1.511381744 |
| 4, member E // 12p13.31 // 26 | | |
| NM_002704 // PPBP // pro-platelet basic protein | 8100971 | 1.5101405 |
| (chemokine (C—X—C motif) ligand | | |
| NM_001657 // AREG // amphiregulin // 4q13-q21 // | 8095744 | 1.508130484 |
| 374 /// BC009799 // AREG // amp | | |

TABLE 3
Short list of genes in PH group

| | |
|---|---|
| NM_006536 // CLCA2 // CLCA family member 2, | 7902702 |
| chloride channel regulator // 1p31-p | |
| NM_175929 // FGF14 // fibroblast growth factor | 7972650 |
| 14 // 13q34 // 2259 /// NM_004115 | |
| NM_000564 // IL5RA // interleukin 5 receptor, | 8085062 |
| alpha // 3p26-p24 // 3568 /// NM_1 | |
| NM_002421 // MMP1 // matrix metallopeptidase 1 | 7951271 |
| (interstitial collagenase) // 11q | |
| NM_001040058 // SPP1 // secreted phosphoprotein 1 // | 8096301 |
| 4q21-q25 // 6696 /// NM_000 | |

TABLE 4
Short list of genes in no-PH group

| | |
|---|---|
| NM_002852 // PTX3 // pentraxin-related gene, rapidly | 8083594 |
| induced by IL-1 beta // 3q2 | |
| NM_000600 // IL6 // interleukin 6 (interferon, beta 2) // | 8131803 |
| 7p21 // 3569 /// ENST0 | |
| NM_002964 // S100A8 // S100 calcium binding protein | 7920244 |
| A8 // 1q21 // 6279 /// ENST0 | |
| NM_001565 // CXCL10 // chemokine (C—X—C motif) | 8101126 |
| ligand 10 // 4q21 // 3627 /// ENS | |
| NM_002164 // INDO // indoleamine-pyrrole 2,3 | 8146092 |
| dioxygenase // 8p12-p11 // 3620 /// | |

### Example 2

Gene expression profiling in the explanted lung from patients with Pulmonary Fibrosis is a better predictor of Primary Graft Dysfunction after lung transplantation than Pulmonary Artery Pressures

Pulmonary fibrosis is a chronic disease causing inflammation of the lungs. In the majority of cases the cause is never found—defined as idiopathic pulmonary fibrosis (IPF). There are five million people worldwide that are affected by this disease and the incidence rate appears to be increasing. Pulmonary hypertension (PH), although can be caused by many other diseases, is also be presented along with IPF. Pulmonary hypertension is prevalent in approximately 30-45% of IPF patients. In addition, PH is often associated with decreased survival in patients with IPF. Eventually, the majority of patients with IPF go on to develop PH. This condition is often fatal. Chest x-rays, electrocardiography, and echocardiography give clues to the diagnosis, but measurement of blood pressure in the right ventricle via catherization and the pulmonary artery is needed for confirmation.

The diagnosis of PH in IPF is often missed due to the lack of specific clinical symptoms. In addition, diagnosis is often delayed by up to 2 years due to general symptomatic overlap with IPF (shortness of breath, exercise limitation etc). There is a clear for an effective biomarker that accurately predicts PH in IPF. To date, several plasma biomarkers have been evaluated, however only Brain Natriuretic peptide (BNP) has been show to be effective in diagnosing patients that present with PH in addition to IPF. However, it is subject to many confound variables such as left heart disease, sex, age and renal dysfunction. This would limit it's effectiveness as a diagnostic biomarker in the general IPF population.

Currently there is no approved therapy for PH when associated with IPF. Given the grave consequences of this condition, treatment of PH could improve functional outcomes and survival. Consequently, managing these patients is not only challenging, but also crucial to keep the patients alive until a potential donor for lung transplant is available.

The current disclosure describes a microarray gene signature of lung biopsies comprising of over 220 genes that can be used to diagnose PH in IPF patients before the onset of further PH complications. Work is in progress to reduce this gene signature to a smaller number of significant genes as well as RT-PCR validation of some of the key genes discovered.

### Secondary Pulmonary Hypertension in IPF

Secondary pulmonary hypertension is defined as a mean Pulmonary Arterial Pressure (mPAP) mmHg. The prevalence is 32-85% (46-85% in patients awaiting lung transplant. There is poor correlation with PFTs, except for DLCO and there is no approved treatment (Nathan S D, et al. Idiopathic Pulmonary Fibrosis and Pulmonary Hypertension: connecting the dots. AMJRCCM 2007; 175: 875 80)

### Possible Mechanisms of Secondary PH

Possible mechanisms include pulmonary artery vasoconstriction, Pulmonary artery remodeling: alveolar damage, abnormal incorporation of connective tissue, ongoing inflammation, vessel ablation, despite pro angiogenic environment and/or abnormal morphology of new vessel formation; endothelial cell dysfunction (Nathan S D, et al. Idiopathic Pulmonary Fibrosis and Pulmonary Hypertension: connecting the dots. AMJRCCM 2007; 175: 875 80).

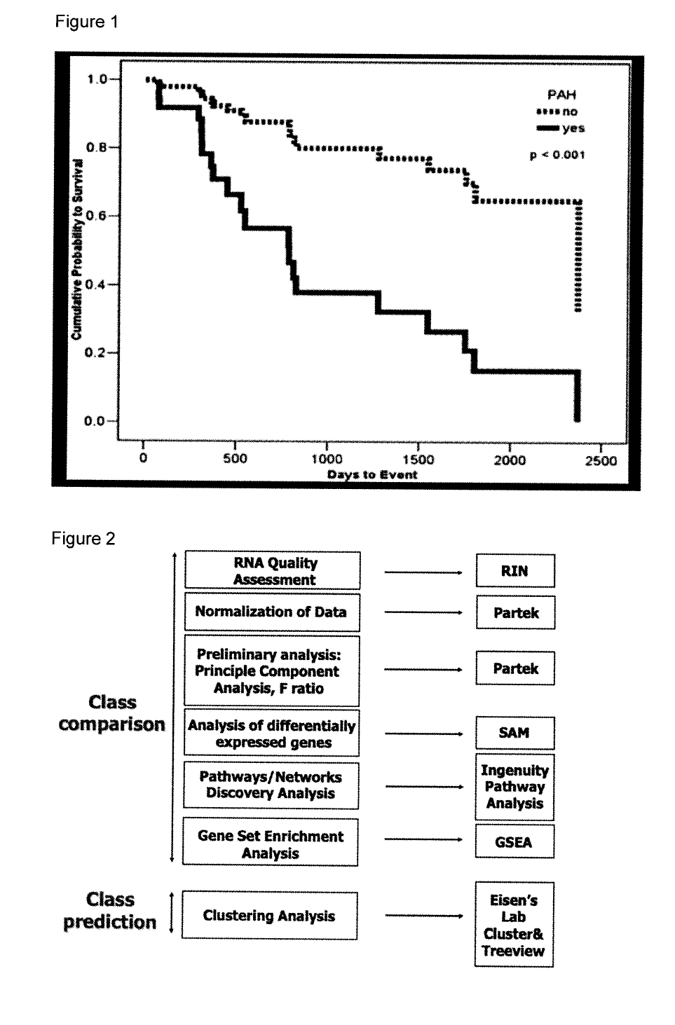

PH has an effect on prognosis (FIG. 1).

It was sought to determine if different gene expression signatures in Pulmonary Fibrosis (PF) patients could be determined based on their pulmonary arterial pressures (PAP)s and to analyze their impact on Primary Graft Dysfunction (PGD) after lung transplantation (LT).

### Methods and Materials.

RNA was extracted from explanted lung in 84 recipients with PF (69 bilateral LT). Demographic data is provided in Tables 5 and 6. PAPs were recorded intraoperatively before starting LT. 17 patients had severe PH (mean PAP≧40 mmHg; PH Group), 22 had low pressures (mPAP<20 mmHg; NoPH Group), and 45 had intermediate mPAP values (21-39 mmHg; Intermediate Group). PGD on arrival in the ICU was defined according to the ISHLT criteria. See FIG. 2 for schematic of method.

### Computation of Probeset Expression Measures

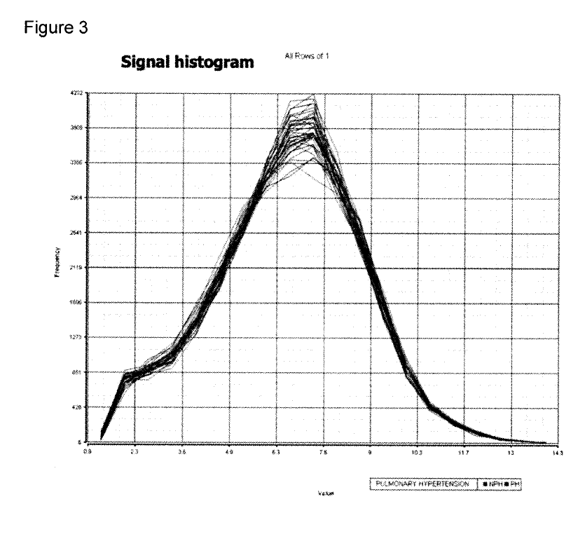

Array platform used for experiments: Human Gene 1.0 Set Array. RMA background correction. Quantile normalization. Summarization within each probe set with the median polish technique, to generate a single measure of expression. Control probes excluded. A signal histogram is provided in FIG. 3.

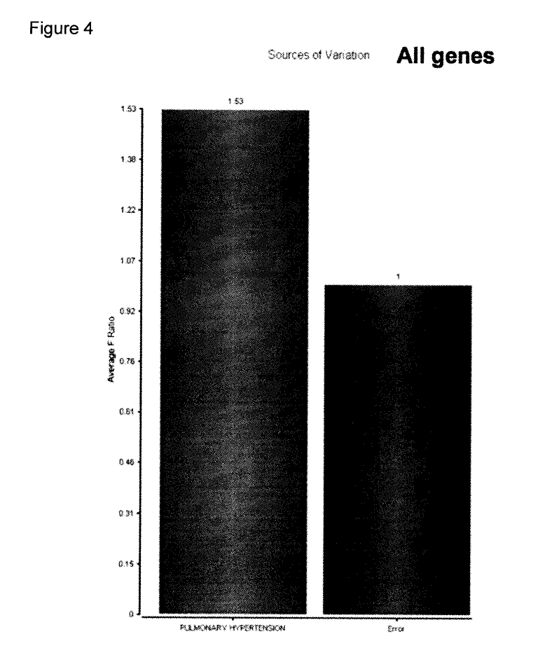

FIG. 4 demonstrates that the microarray quality was good.

### SAM Analysis-Detection of Differentially Expressed Genes

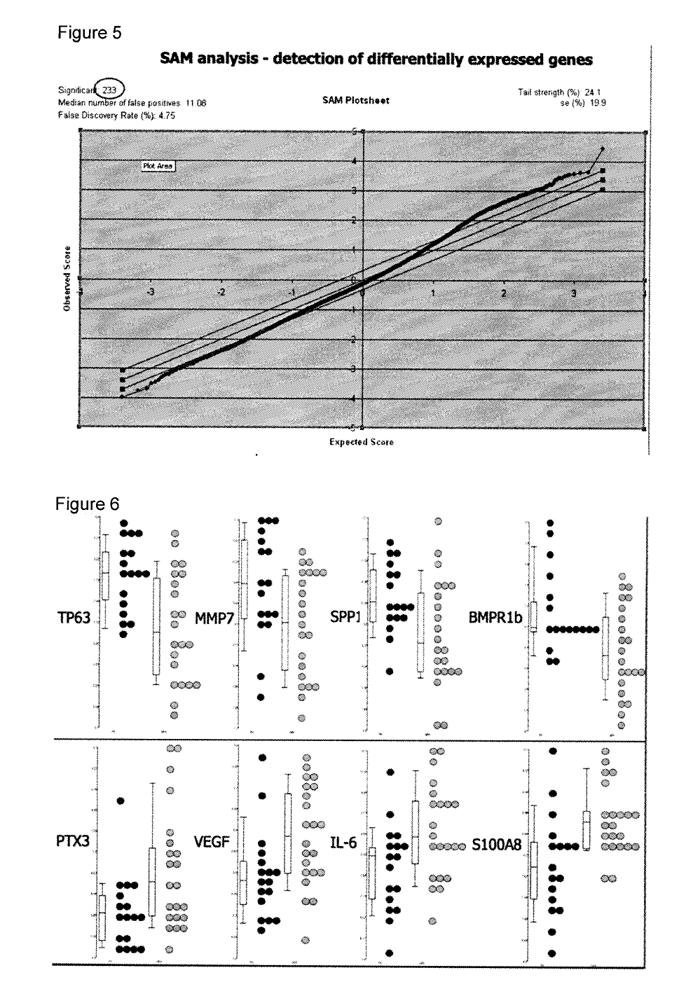

Control probe sets excluded. 28869 probe sets used for analysis. Criteria: FDR* q value <0.05 & fold change M 0.5. A plot based on SAM analysis is provided in FIG. 5.

### Results.

PH patients exhibited an increased expression of genes, gene sets and networks related with myofibroblasts proliferation and vascular remodeling, including Osteopontin, MMP7, MMP13, BMPR1b. NoPH patients showed a strong expression of pro-inflammatory genes, including IL-6, PTX3, S100A8, VEGF.

mPAP did not predict PGD. However, two distinct gene signatures were observed in PH and noPH groups. In the Intermediate group, two-dimensional hierarchical clustering based on the 233 differentially expressed genes (PH vs. NoPH groups) dichotomized patients into two distinct subgroups. Patients clustered in the subgroup with increased expression of NoPH-related genes had a higher incidence of PGD II-III (52% vs. 14%, p=0.006). Looking at the whole population, NoPH-related gene signature was associated with a higher incidence of PGD II-III when compared to the PH-related gene signature (40% vs. 17%; p=0.022). A logistic regression model in the whole population showed that clustering algorithm based on PH vs NoPH gene signature was the only significant predictor of PGD (Chi square 5.6, p=0.017), while mPAP and type of operation were not.

Analysis using ingenuity analysis found genes to be up or down regulated in the PH group and the No PH group including genes involved in ECM remodeling and the inflammatory response.

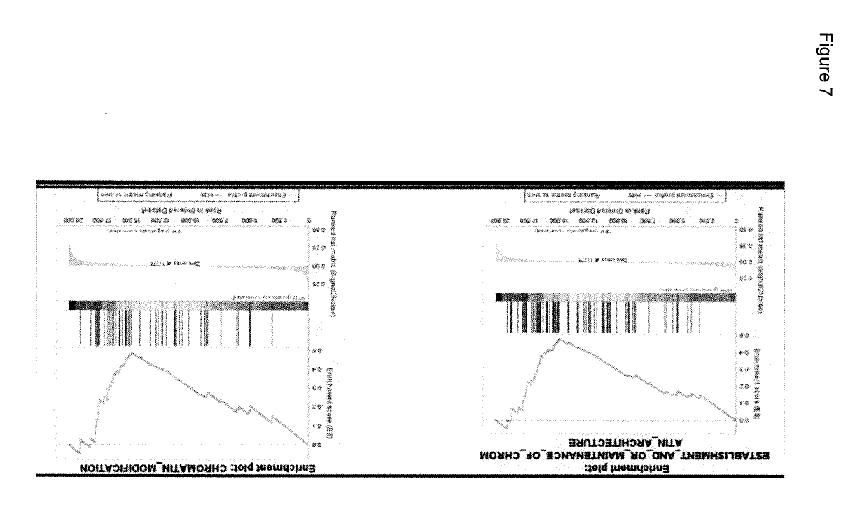

The top 20 genes upregulated in the PH group is provided in Table 7. Upregulated gene in the PH group involved in the ECM remodeling based on ingenuity pathway analysis is provided in Table 8. The top 10 genes upregulated in the No PH group are provided in Table 9. Genes upregulated in the No PH group involved in the inflammatory response based on ingenuity analysis are provided in Table 10. FIG. 6: examples of levels of gene expression for some specific genes.

The genes were also analysed by gene set enrichment analysis. GSEA is a computational method that determines whether an a priori defined set of genes shows statistically significant concordant differences between two biological states. GSEA derives its power by focusing on gene sets, that is groups of genes that share common biological function, chromosomal location, or regulation (Subramanian A et al. Gene set enrichment analysis: a knowledge-based approach for interpreting genome-wide expression profiles. PNAS 2005; 102: 15545-50). Looking at FIG. 7 the score at the peak of the plot is the ES for the gene set. Gene sets with a distinct peak at the beginning or end of the ranked list are generally the most interesting. The middle panel indicates where the members of the gene set appear in the ranked list of genes. For a positive ES the leading edge subset is the set of members that appear in the ranked list prior to the peak score. The C5 GO gene set database was analysed. Upregulated gene sets in the PH group are listed in Table 11.

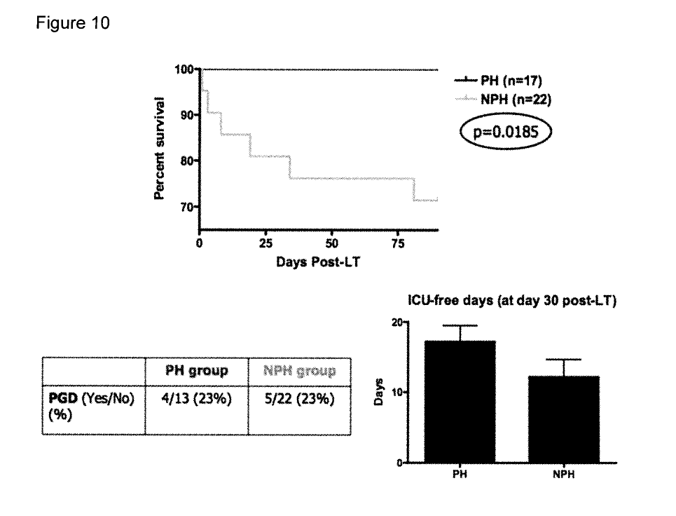

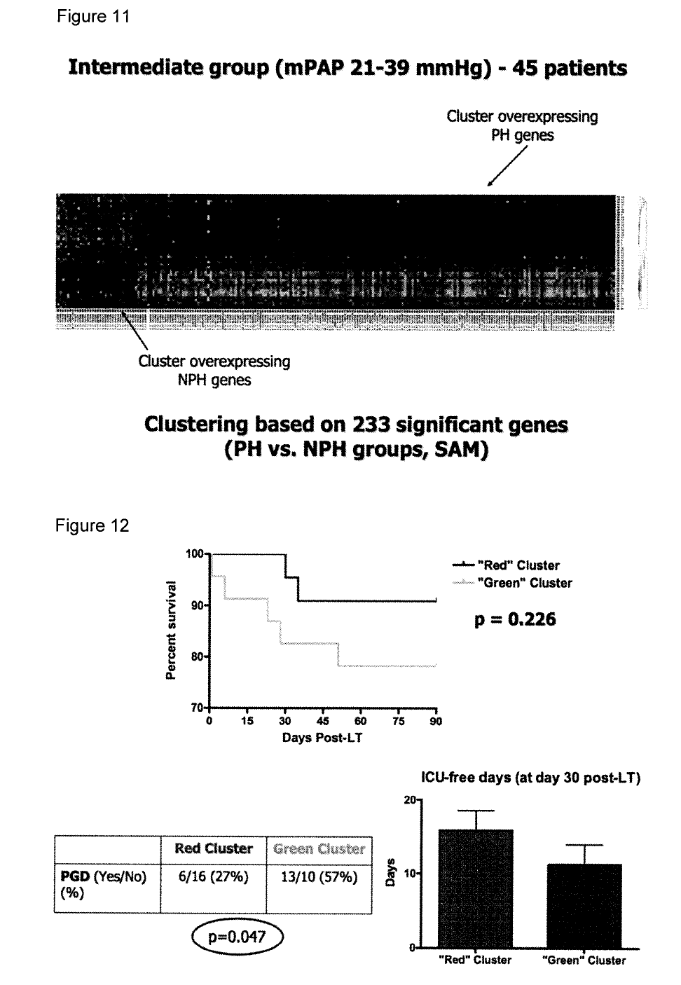

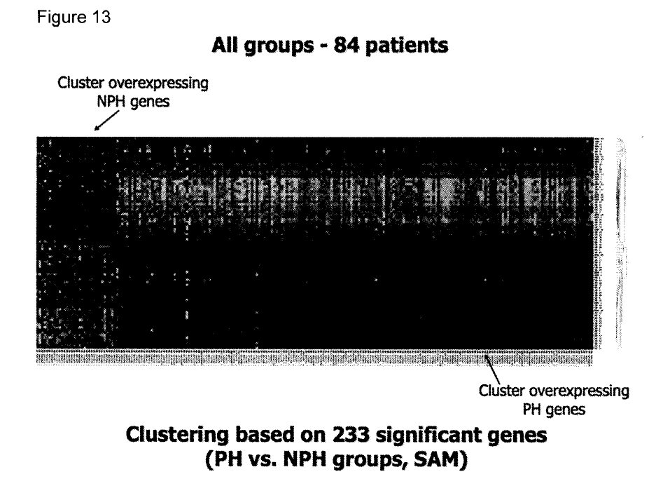

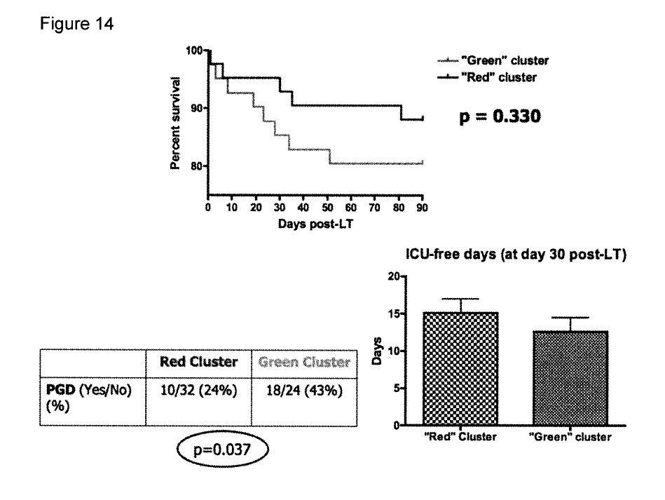

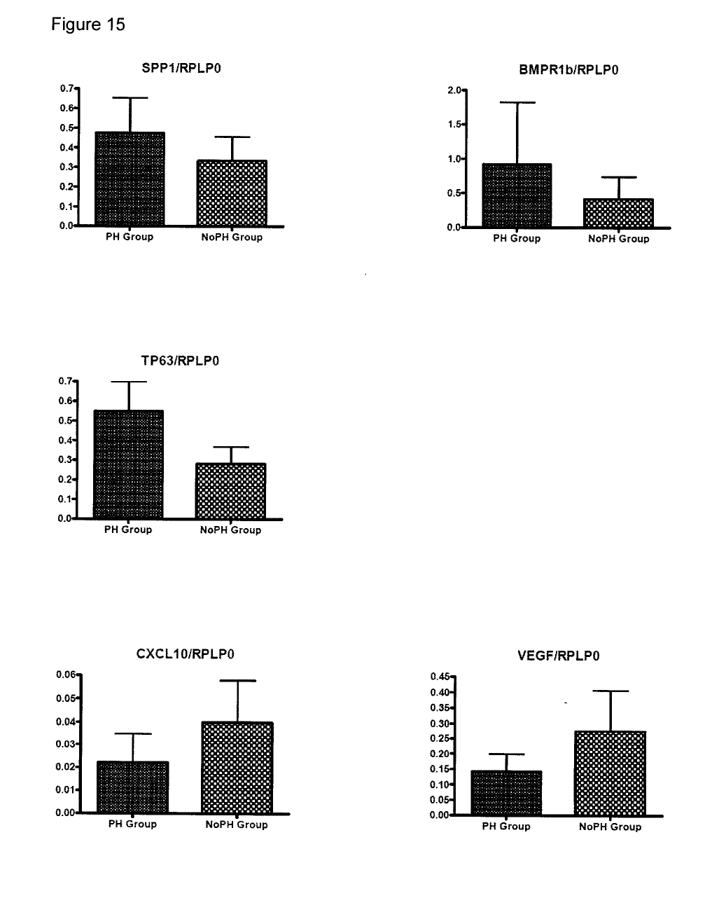

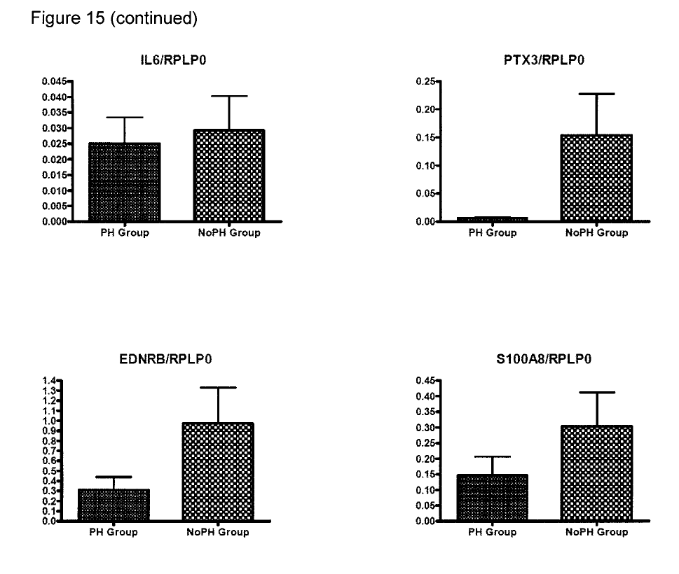

Clustering analysis was performed and results are described in FIGS. 9-14 and Tables 12 and 13.

### Conclusions

PH and NPH groups of PF patients exhibit distinct gene expression profiles

Genetic predisposition, increased proliferation of fibroblasts, disruption of BM and endothelial cell death may be the leading events in the PH phenotype

The pro pro-inflammatory gene signature of NPH patients shows an association with post post-transplant outcome.

Although PAP value is not a predictor of PGD, PF patients exhibit two distinct gene expression profiles associated with different risk of PGD post-LT.

TABLE 5
Demographic and functional characteristics of patients (n = 84)

| Variable | Average ± SD |
|---|---|
| Age (years) | 59 ± 8 |
| Gender (male/female) (% males) | 52/32 (62%) |
| BMI (kg/m²) | 26 ± 4 |
| UIP/Non-UIP diagnosis (% UIP) | 64/20 (76%) |
| Transplant (Single/Bilateral) (% Bilateral) | 15/69 (78%) |
| Cardio-pulmonary Bypass (Yes/No) | 54/30 (64%) |
| ICU stay (days) (all patients) | 17 ± 17 |
| ICU-free days (at day 30 post-LT) | 14 ± 12 |
| Deaths in the ICU | 13 (15%) |
| FVC (% pred) | 54 ± 18 |
| DLCO (% pred) | 41 ± 15 |
| TLC (% pred) | 61 ± 14 |
| 6-min Walking Distance (m) | 295 ± 94 |
| mPAP (mmHg) | 29 ± 12 |
| Presence of Pulmonary Hypertension (Yes/No) | 52/32 (62%) |
| Severe Pulmonary Hypertension (≧40 mmHg) (Yes/No) | 17/67 (20%) |

TABLE 6
Demographic and functional characteristics of patients
for PH and NO PH groups

| | PH group | NO PH group | |
|---|---|---|---|
| | mPAP ≧ 40 | mPAP ≦ 20 | |
| Variable | (n = 17) | (n = 22) | p value |
| Age (years) | 58 ± 8 | 61 ± 8 | n.s. |
| Gender (M/F) | 11/6 | 11/11 | n.s. |
| (% males) | (65%) | (50%) | |
| BMI (kg/m²) | 26 ± 4 | 25 ± 4 | n.s. |
| UIP/Non-UIP diagnosis | 13/4 | 14/8 | n.s. |
| (% UIP) | (76%) | (64%) | |
| Transplant | 13/4 | 18/4 | n.s. |
| (Single/Bilateral) (% | (76%) | (82%) | |
| Single) | | | |
| Cardio-pulmonary | 15/2 | 12/10 | n.s. |
| Bypass (Yes/No) (%) | (88%) | (55%) | |
| ICU stay (days) | 13 ± 10 | 14 ± 13 | n.s. |
| FVC (% pred) | 61 ± 24 | 48 ± 15 | n.s. |
| TLC (% pred) | 65 ± 18 | 58 ± 15 | n.s. |
| DLCO (% pred) | 27 ± 9 | 59 ± 20 | 0.002 |
| 6MWD (m) | 271 ± 91 | 258 ± 118 | n.s. |
| mPAP (mmHg) | 48 ± 9 | 17 ± 2 | <0.0001 |

TABLE 7
Top 10 genes upregulated in the PH group

| | Gene | | | Fold | FDR |
|---|---|---|---|---|---|
| Rank | Symbol | Gene name | d | change | q value |
| 1 | CLCA2 | CLCA family member 2, | 3.46 | 2.4 | <0.0001 |
| | | chloride channel regulator | | | |
| 2 | C1orf168 | Chromosome 1 open | 3.44 | 1.98 | <0.0001 |
| | | reading frame 168 | | | |
| 3 | ABCB1 | ATP-bindng cassette, sub- | 3.23 | 1.61 | <0.0001 |
| | | family B | | | |
| 4 | Unknown | Unknown | 3.21 | 1.54 | <0.0001 |
| 5 | Unknown | Unknown | 3.12 | 1.56 | <0.0001 |
| 6 | DSP | Desmoplakin | 3.08 | 1.54 | <0.0001 |
| 7 | SLITRK6 | SLIt and NTRK-like | 3.08 | 1.84 | <0.0001 |
| | | family, member 6 | | | |
| 8 | FGF14 | Fibroblast Growth | 3.07 | 1.50 | <0.0001 |
| | | Factor 14 | | | |
| 9 | CCDC81 | Coilder-coil domain | 3.07 | 1.68 | <0.0001 |
| | | containing 81 | | | |
| 10 | CHST9 | Carbohydrate (N- | 3.05 | 2.32 | <0.0001 |
| | | acetylgalactosamine 4·0) | | | |
| | | sulfotransferase | | | |

TABLE 8
Upregulated genes in the PH group involved in the ECM remodeling
(Ingenuity Pathway Analysis)

| | Gene | | | Fold | FDR |
|---|---|---|---|---|---|
| Rank | Symbol | Gene Name | d | change | q value |
| 160 | MMP1 | Matrix metallopeptidase 1 | 2.28 | 2.11 | 0.010 |
| 168 | MMP13 | Matrix metallopeptidase 13 | 2.20 | 1.66 | 0.014 |
| 174 | SPP1 | Secreted phosphoprotein 1 | 2.18 | 1.94 | 0.014 |
| | | (Osteopontin) | | | |
| 184 | MMP7 | Matrix metallopeptidase 7 | 2.12 | 1.62 | 0.014 |

TABLE 9
Top 10 genes upregulated in the NPH group

| | Gene | | | Fold | FDR |
|---|---|---|---|---|---|
| Rank | Symbol | Gene name | d | change | q value |
| 1 | IRF1 | Interferon Regulatory | −3.76 | −1.55 | <0.0001 |
| | | Factor 1 | | | |
| 2 | GLDN | Gliomedin | −3.11 | −1.95 | 0.033 |
| 3 | INDO | Indoleamine-pyrrole 2,3 | −3.00 | −2.01 | 0.033 |
| | | dioxygenase | | | |
| 4 | MT1A | Metallothionein 1A | −2.94 | −1.58 | 0.033 |
| 5 | ANKRD1 | Ankyrin repeat domain 1 | −2.92 | −2.56 | 0.033 |
| 6 | S100A8 | S100 calcium binding | −2.90 | −1.73 | 0.033 |
| | | protein A8 | | | |
| 7 | IL18RAP | Interleukin 18 receptor | −2.86 | −1.70 | 0.033 |
| | | accessory protein | | | |
| 8 | GBP4 | Guanylate binding protein 4 | −2.84 | −1.55 | 0.033 |
| 9 | CD274 | CD274 molecule | −2.80 | −1.60 | 0.033 |
| 10 | SOCS3 | Suppressor of cytokine | −2.72 | −1.62 | 0.033 |
| | | signaling 3 | | | |

TABLE 10
Upregulated genes in the NPH group involved in the
inflammatory response (Ingenuity Pathway Analysis)

| | Gene | | | Fold | FDR |
|---|---|---|---|---|---|
| Rank | Symbol | Gene Name | d | change | q value |
| 6 | S100A8 | S100 calcium binding | −2.89 | 1.73 | 0.025 |
| | | protein A8 | | | |
| 7 | IL18RAP | Interleukin 18 receptor | −2.86 | 1.70 | 0.025 |
| | | accessory protein | | | |
| 10 | SOCS3 | Suppressor of cytokine | −2.72 | 1.62 | 0.025 |
| | | signaling 3 | | | |
| 14 | CXCL10 | Chemokine (C—X—C | −2.49 | 1.87 | 0.035 |
| | | motif) ligand 10 | | | |
| 15 | IL6 | Interleukin 6 | −2.41 | 1.91 | 0.035 |
| 16 | CXCL11 | Chemokine (C—X—C | −2.39 | 1.69 | 0.035 |
| | | motif) ligand 11 | | | |
| 18 | CXCL9 | Chemokine (C—X—C | −2.37 | 1.65 | 0.035 |
| | | motif) ligand 9 | | | |
| 19 | PTX3 | Long Pentraxin 3 | −2.36 | 1.93 | 0.035 |
| 22 | S100A12 | S100 calcium bindin | −2.29 | 1.53 | 0.035 |
| | | g protein A12 | | | |
| 26 | CXCL2 | Chemokine (C—X—C | −2.18 | 1.53 | 0.038 |
| | | motif) ligand 2 | | | |
| 31 | SERPINE1 | Serpin peptidase | −1.95 | 1.59 | 0.041 |
| | | inhibitor, clade E | | | |
| 34 | PPBP | Pro-platelet basic | −1.71 | 1.51 | 0.051 |
| | | protein | | | |
| | VIPR1 | Vasoactive intestinal | −1.97 | 1.42 | 0.041 |
| | | peptide receptor 1 | | | |
| | VEGF-A | Vascular endothelial | −2.09 | 1.21 | 0.038 |
| | | gorwth factor A | | | |
| | EDNRB | Endothelin receptor | −1.82 | 1.21 | 0.048 |
| | | type B | | | |
| | TGFb1 | Transforming growth | −1.90 | 1.12 | 0.041 |
| | | factor, beta 1 | | | |

TABLE 11
Upregulated gene sets in PH group

| | | NOM p | FDR q |
|---|---|---|---|
| GENE SET | NES | value | value |
| ESTABLISHMENT AND OR | −2.10 | 0.000 | 0.022 |
| MAINTENANCE OF CHROMATIN | | | |
| ARCHITECTURE | | | |
| CHROMATIN MODIFICATION | −2.00 | 0.004 | 0.035 |
| CHROMOSOME ORGANIZATION AND | −1.96 | 0.002 | 0.040 |
| BIOGENESIS | | | |
| MICROTUBULE ORGANIZING CENTER | −1.93 | 0.000 | 0.047 |
| PART | | | |

TABLE 12

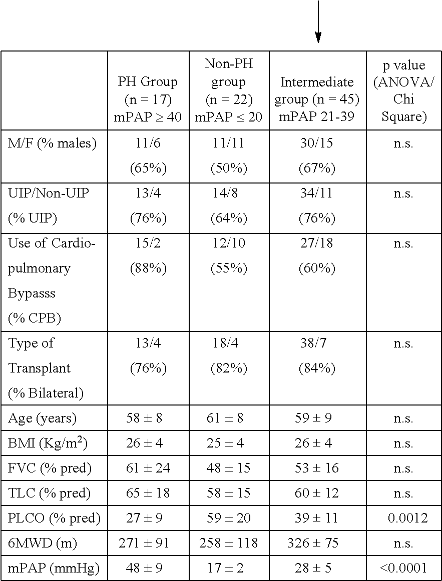

TABLE 13
Ordinal Logistic Regression Model for the prediction of PGD
incidence. p value of the model = 0.025

| Independent | | |
|---|---|---|
| Variable | Chi Square | p value |
| Cardio-Pulmonary | 4.52 | [symbol] |
| Bypass | | |
| Clustering | 4.57 | [symbol] |
| Type of Transplant | 2.20 | 0.333 |

### Example 3

Gene expression levels of selected genes were assessed by RT-PCR. PTX3 was one of the gene expression levels measured by RT-PCR. The levels were elevated in the noPH group and absent in the PH group.

### Example 4

An illustration of a use of this technology in the clinic is as follows: A patient is diagnosed as having pulmonary fibrosis by a clinician. At biopsy or at surgery, a tissue sample is removed, processed and the relative expression levels of 5 or more genes listed in Table 1, 2, 3, 4 7, 8, 9, and/or 10 are measured.

If the expression profile is similar to the PH profile, the subject is considered to have a probability of clinical disease and/or PGD similar to the PH class and the patient is considered to have a good outcome or be at a decreased risk of PGD.

If the expression profile is similar to the no-PH profile, the subject is considered to have a probability of clinical disease and/or PGD similar to the no-PH class and the patient is considered to have a poor outcome or be at a increased risk of PGD.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. All sequences (e.g. nucleotide, including RNA and cDNA, and polypeptide sequences) of genes listed in the tables such as Table 1 and/or 2, for example referred to by accession number are herein incorporated specifically by reference.

1. A method of classifying a subject with pulmonary fibrosis (PF) comprising:
a. measuring a gene expression level of a plurality of genes, comprising at least 1 for example 5 genes, selected from Table 1, 2, 3, 4 7, 8, 9, and/or 10 in a sample taken from the subject; and
b. classifying the subject as having a PH subtype when the expression levels of the plurality of genes is most similar to a PH profile and classifying the subject as a noPH subtype when the expression levels of the plurality of genes is most similar to a noPH profile.

2. The method of claim 1 wherein an increased expression of 5 or more genes in Table 7 classifies the subject has a PH subtype and/or an increased expression of 5 or more genes from Table 9 classifies the subject as a noPH subtype.

3.-5. (canceled)

6. The method of claim 1, the method comprising:
I.
a. determining an expression profile by measuring the gene expression levels of a plurality of genes, comprising at least 5 genes, selected from a Table 1, 3 or 7, in a sample from the subject; and
b. classifying the subject as having a good prognosis or a poor prognosis based on the expression profile;
wherein increased expression of the 5 or more genes is indicative that the subject is a noPH subtype and has a poor prognosis post lung transplant; and/or
II.
a. determining an expression profile by measuring the gene expression levels of a plurality of genes, comprising at least 5 genes, selected from a Table 2, 4 or 9, in a sample from the subject; and
b. classifying object as having a good prognosis or a poor prognosis based on the expression profile
wherein increased expression of the 5 or more genes is indicative that the subject is a PH subtype and has a good prognosis post lung transplant.

7. (canceled)

8. The method of claim 6, the method comprising:
a. calculating a first measure of similarity between a first expression profile and a good prognosis reference profile and a second measure of similarity between the first expression profile and a poor prognosis reference profile; the first expression profile comprising the expression levels of a first plurality of genes in a sample of the subject; the good prognosis reference profile comprising, for each gene in the first plurality of genes, the average expression level of the gene in a plurality of good prognosis subjects; and the poor prognosis reference profile comprising, for each gene in the first plurality of genes, the average expression level of the gene in a plurality of poor prognosis subjects, the first plurality of genes comprising at least 5 of the genes listed in Table 1, 2, 3, 4 7, 8, 9, and/or 10; and
b. classifying the subject as having a good prognosis if the first expression profile has a higher similarity to the good prognosis reference profile than to the poor prognosis reference profile, or classifying the subject as poor prognosis if the first expression profile has a higher similarity to the poor prognosis reference profile than to the good prognosis reference profile.

9. The method of claim 2, the method comprising:
a. calculating a first measure of similarity between a first expression profile and a PF PH subtype reference profile and a second measure of similarity between the first expression profile and a PF noPH subtype reference profile; the first expression profile comprising the expression levels of a first plurality of genes in a sample of the subject; the PF PH subtype reference profile comprising, for each gene in the first plurality of genes, the average expression level of the gene in a plurality of PF PH subtype subjects; and the PF noPH subtype reference profile comprising, for each gene in the first plurality of genes, the average expression level of the gene in a plurality of PF noPH subtype subjects, the first plurality of genes comprising at least 5 of the genes listed in Tables 7 and 9; and
b. classifying the subject as having a PF PH subtype if the first expression profile has a higher similarity to the PF PH subtype reference profile than to the PF noPH subtype reference profile, or classifying the subject as PF noPH subtype if the first expression profile has a higher similarity to the PF noPH subtype reference profile than to the PF PH subtype reference profile.

10. A method of claim 1 for classifying a subject having PF as having a PH subtype or no-PH subtype; and/or a good prognosis or a poor prognosis, the method comprising:
a. calculating a measure of similarity between an expression profile and one or more subtype and/or prognosis reference profiles, the expression profile comprising the expression levels of a first plurality of genes in a sample taken from the subject; the one or more subtype and/or prognosis reference profiles comprising, for each gene in the plurality of genes, the average expression level of the gene in a plurality of subjects associated with the subtype and/or prognosis reference profile, for example a good prognosis reference profile and/or poor prognosis reference profile; the plurality of genes comprising at least 5 of the genes listed in Table 7, 8, 9, and/or 10; and
b. classifying the subject as having the PH subtype and/or a good prognosis if the expression profile has a high similarity to the PH subtype and/or the good prognosis reference profile or has a higher similarity to the to the PH subtype and/or the good prognosis reference profile than to the PH poor prognosis reference profile or classifying the subject as having the noPH subtype and/or poor prognosis if the expression profile has a low similarity to the PH subtype and/or the good prognosis reference profile or has a higher similarity to the noPH subtype and/or the poor prognosis reference profile than to the PH subtype and/or good prognosis reference profile; wherein the expression profile has a high similarity to the PH subtype and/or the good prognosis reference profile if the similarity to the PH subtype and/or the good prognosis reference profile is above a predetermined threshold, or has a low similarity to the PH subtype and/or the good prognosis reference profile if the similarity to the PH subtype and/or good prognosis reference profile is below the predetermined threshold.

11. The method of claim 1, further comprising displaying or outputting to a user interface device, a computer readable storage medium, or a local or remote computer system, the classification produced by the classifying step (b).

12. A computer-implemented method for determining a prognosis of a subject having PF comprising: classifying, on a computer, the subject as having a good prognosis or a poor prognosis based on an expression profile comprising measurements of expression levels of a plurality of genes in a sample from the subject, the plurality of genes, comprising at least 5 genes listed in Table 1, 2, 3, 4 7, 8, 9, and/or 10, according to the method of claim 1; wherein a good prognosis predicts a decreased risk of PGD post lung transplant, and wherein a poor prognosis predicts an increased risk of PGD post lung transplant.

13. The method of claim 8, wherein the reference profile(s) is pre-generated, and for example comprised in a database, or wherein the reference profile(s) is generated de novo.

14. (canceled)

15. The method of claim 13, wherein the method comprises:
I.
a. generating a good prognosis reference profile;
b. generating a poor prognosis reference profile;
c. generating a first expression profile of a subject with PH;
d. calculating a measure of similarly between the first expression profile and one or more of good prognosis reference profiles; and
e. classifying the subject as having a good prognosis if the first expression profile is similar, or has higher similarity, to the good prognosis reference profile and/or classifying the subject as having a poor prognosis if the first expression profile is similar, or has a higher similarity to the poor prognosis reference profile; and/or
II.
a. generating a PH subtype profile reference profile;
b. generating a no PH reference profile;
d. calculating a measure of similarly between the first expression profile and one or more of the PH subytpe reference profile and
e. classifying the subject as having a PH subtype if the first expression profile is similar, or has higher similarity, to the PH subtype reference profile and/or classifying the subject as haying a noPH subtype if the first expression profile is similar, or has a higher similarity to the noPH subtype reference profile.

16.-17. (canceled)

18. The method of claim 1, wherein the gene set or plurality of genes comprises at least 6, 7, 8, 9, 10, 11, 12, 13, 14 or 15 genes listed in Table 1, 2, 3, 4 7, 8, 9, and/or 10.

19. (canceled)

20. The method of claim 1, wherein the gene set or plurality of genes comprises or consists of the genes listed in Table 1, 2, 3, 4 7, 8, 9, and/or 10, preferably consists of the genes listed in Table 7 and/or 9.

21. The method of claim 1, wherein the subject is in a clinical trial and/or the method is for selecting subjects for a clinical trial.

22. (canceled)

23. A method of selecting or optimizing a PF or PGD treatment and/or treating a PF subject comprising:
a. determining a subject gene expression profile and prognosis according to claim 1; and
b. selecting a treatment indicated by their prognosis and/or treating the subject with a treatment indicated by their prognosis.

24.-26. (canceled)

27. The method of claim 1, wherein the method comprises first obtaining the sample from the subject, optionally wherein the sample comprises a surgical resection, or a biopsy.

28.-29. (canceled)

30. The method of claim 1, wherein determining the expression profile comprises contacting the sample with an analyte specific reagent (ASR).

31. (canceled)

32. A method of selecting a human subject for inclusion or exclusion in a clinical trial, the method comprising:
a. classifying a subject as a PF PH subtype or a PF noPH subtype according to the method of claim 1; and
b. including or excluding the subject if the expression level and/or profile indicates that the subject has a PF PH subtype or a PF noPH subtype.

33. (canceled)

34. A computer system comprising:
a. a database including records comprising reference expression profiles associated with clinical outcomes, each reference profile comprising the expression levels of a plurality of genes listed in Table 1, 2, 3, 4 7, 8, 9, and/or 10;
b. a user interface capable of receiving and/or inputting a selection of gene expression levels of a plurality of genes, the plurality comprising at least 5 genes listed in Table 1, 2, 3, 4 7, 8, 9, and/or 10, for use in comparing to the gene reference expression profiles in the database;
c. an output that displays a prediction of clinical outcome according to the expression levels of the plurality of genes, determined according to the method of claim 1.

35. A method for identifying candidate agents for use in treatment of PF and/or PGF comprising:
a. obtaining an expression level for at least 5 genes listed in Table 1, 2, 3, 4 7, 8, 9, and/or 10 in a first test sample of a lung cell or a population of cells comprising lung cells, wherein the cell or population of cells is optionally in vitro or in vivo;
b. contacting for example, by incubating, the cell or population of cells with a test agent;
c. obtaining an expression level for the at least 5 genes in a second test sample, wherein the second test sample is obtained subsequent to incubating the cell culture with the test agent;
d. comparing the expression level of the at least 5 genes in the first and second test samples to a good prognosis reference expression profile and a poor prognosis reference expression profile of the at least 5 genes;
wherein a change in the expression level of the genes in the second sample indicating a greater similarity to a good prognosis reference profile indicates that the agent is a candidate therapeutic.

36. A composition comprising a plurality of ASRs, optionally probes or primers, for determining expression of a plurality of genes being at least 5 of the genes listed in Table 1, 2, 3, 4 7, 8, 9, and/or 10.

37. (canceled)

38. An array comprising for each gene in a plurality of genes, the plurality of genes being at least 5 of the genes listed in Table 1, 2, 3, 4 7, 8, 9, and/or 10, one or more polynucleotide probes complementary and hybridizable to a coding sequence in the gene or the composition of claim 36.

39. A kit for determining prognosis in a subject having PF comprising:
I.
a. the array of claim 38;
b. one or more of specimen collector and RNA preservation solution; and optionally
c. instructions for use; or
II.
a. a plurality of ASRs, optionally a plurality of probes comprising at least two probes, wherein each probe hybridizes and/or is complementary to a nucleic acid sequence corresponding to a gene selected from Table 1, 2, 3, 4 7, 8, 9, and/or 10; and optionally
b. one or more of specimen collector RNA preservation solution and instructions for use; or
III.
a. a plurality of antibodies comprising at least two antibodies, wherein each antibody of the set is specific for a polypeptide corresponding to a Gene selected from Table 1, 2, 3, 4 7, 8, 9, and/or 10; and optionally
b. one or more of specimen collector, polypeptide preservation solution and instructions for use.

40.-41. (canceled)
